(19) United States
(12) Patent Application Publication
(10) Pub. No.: US 2021/0008564 A1
(43) Pub. Date: Jan. 14, 2021

(54) ANALYSIS DEVICE, CARTRIDGE, ANALYSIS SYSTEM AND METHOD FOR TESTING A SAMPLE

(71) Applicant: BOEHRINGER INGELHEIM VETMEDICA GMBH, Ingelheim am Rhein (DE)

(72) Inventor: Hannah SCHMOLKE, Didderse (DE)

(73) Assignee: BOEHRINGER INGELHEIM VETMEDICA GMBH, Ingelheim am Rhein (DE)

(21) Appl. No.: 16/979,552

(22) PCT Filed: Apr. 2, 2019
(86) PCT No.: PCT/EP2019/058293

§ 371 Date: Sep. 10, 2020

(30) Foreign Application Priority Data
Apr. 6, 2018 (EP) 18166137.2

(51) Int. Cl. B01L 9/00; B01L 3/00; G01N 15/05; G01F 23/292; G01N 1/14

(52) U.S. Cl. CPC B01L 9/527; B01L 3/502715; G01N 15/05; B01L 2300/0867; G01N 1/14; B01L 2200/04; B01L 2300/0663; G01F 23/292

(57) ABSTRACT

An analysis device, a cartridge, an analysis system and a method for preferably testing a biological sample. The analysis system has a sample sensor for monitoring the sample in a receiving cavity of the cartridge. The sample sensor preferably allows sedimentation of the sample to be measured or detected.

### BACKGROUND OF THE INVENTION

### Field of the Invention

The present invention relates to an analysis device for testing a sample by means of a cartridge, to a cartridge for testing a sample in an analysis device, to an analysis system comprising an analysis device and a cartridge for receiving and testing a sample and to a method for testing a sample with an analysis system comprising an analysis device and a cartridge.

Preferably, the present invention deals with analyzing and testing a sample, in particular from a human or animal, particularly preferably for analytics and diagnostics, for example with regard to the presence of diseases and/or pathogens and/or for determining blood counts, antibodies, hormones, steroids or the like. Therefore, the present invention is in particular within the field of bioanalytics. A food sample, environmental sample or another sample may optionally also be tested, in particular for environmental analytics or food safety and/or for detecting other substances.

Preferably, by means of the present invention, at least one analyte (target analyte) of a sample can be determined, detected or identified. In particular, the sample can be tested for qualitatively or quantitatively determining at least one analyte, for example in order for it to be possible to detect or identify a disease and/or pathogen.

Preferably, by means of the present invention, nucleic-acid sequences, in particular DNA sequences and/or RNA sequences, can be determined, detected or identified as analytes of a sample, or proteins, in particular antigens and/or antibodies, can be determined, detected or identified as analytes of the sample. More particularly preferably, the present invention deals with systems, devices and other apparatuses for carrying out a nucleic-acid assay for detecting or identifying a nucleic-acid sequence or a protein assay for detecting or identifying a protein.

The present invention deals in particular with what are known as point-of-care systems, i.e., in particular with mobile systems, devices and other apparatuses, and deals with methods for carrying out tests on a sample at the sampling site and/or separately or away from a central laboratory or the like. Preferably, point-of-care systems can be operated autonomously of or independently from a mains network for supplying electrical power.

### Description of the Related Art

U.S. Pat. No. 5,096,669 discloses a point-of-care system for testing a biological sample, in particular a blood sample. The system comprises a single-use cartridge and an analysis device. Once the sample has been received, the cartridge is inserted into the analysis device in order to carry out the test. The cartridge comprises a microfluidic system and a sensor apparatus comprising electrodes, which apparatus is calibrated by means of a calibration liquid and is then used to test the sample.

Furthermore, International Patent Application Publication WO 2006/125767 A1 and corresponding U.S. Pat. No. 9,110,044 B2 disclose a point-of-care system for integrated and automated DNA or protein analysis, comprising a single-use cartridge and an analysis device for fully automatically processing and evaluating molecular-diagnostic analyses using the single-use cartridge. The cartridge is designed to receive a sample, in particular blood, and in particular allows cell disruption, PCR and detection of PCR amplification products, which are bonded to capture molecules and provided with a label enzyme, in order for it to be possible to detect bonded PCR amplification products or nucleic-acid sequences as target analytes in what is known as a redox cycling process.

### SUMMARY OF THE INVENTION

The problem addressed by the present invention is to make possible improved analysis or testing of a sample, in particular comprehensive, efficient, rapid, reliable, hygienic, robust and/or precise testing of the sample being allowed or facilitated.

The above problem is solved by an analysis device, a cartridge, an analysis system or by a method as described herein.

A proposed analysis device is designed for testing, in particular, a biological sample by means of a cartridge. In a preferred example, the biological sample is blood.

The analysis device is preferably designed to receive, position and/or hold the cartridge. Preferably, the cartridge can be held in a clamped manner and/or can be mechanically, electrically, thermally, fluidically and/or pneumatically connected to the analysis device.

The cartridge preferably comprises a receiving cavity for receiving or taking up the sample.

The analysis device preferably comprises a sample sensor for monitoring the sample in the receiving cavity. This allows for monitoring or measuring a state of the sample in the cartridge or receiving cavity, in particular, before testing or further processing of the sample is started. In this way, a fast, effective and/or efficient testing or analysis of the sample can be achieved.

The sample sensor is preferably designed to measure or detect a filling level of the sample or components thereof in the receiving cavity. As an alternative or in addition, the sample sensor is designed to measure or detect a degree of separation or sedimentation of the sample or components thereof in the receiving cavity. This is conducive to a fast, reliable and/or efficient testing of the sample or a component thereof. Components of the sample can in particular be blood cells on the one hand and blood plasma or blood serum on the other hand.

The sample sensor is preferably designed to detect a finished or sufficient sedimentation of the sample. In addition, or as an alternative, the sample sensor is designed to measure or detect a supernatant of the sample, in particular an at least substantially clear and/or colorless liquid such as blood plasma, blood serum or the like. This is conducive to a fast, reliable and/or efficient testing of the sample or a component thereof.

Preferably, the sample sensor is designed to detect or measure electromagnetic radiation, in particular, (visible) light and/or infrared radiation. Particularly, the sample sensor is formed by or comprises a sensor or detector for detecting electromagnetic radiation, in particular an optical and/or infrared sensor. This is conducive to a fast, reliable and/or cost-efficient monitoring of the sample or a component thereof.

The sample sensor is preferably designed to monitor the sample by a reflection measurement, in particular wherein the sample sensor is designed to generate, send, receive and/or measure or detect electromagnetic radiation. Particularly preferably, the analysis device or sample sensor is designed to gain information about the state of the sample by measuring and analyzing reflected electromagnetic radiation which has been generated by the analysis device or sample sensor. This is conducive to a fast, reliable and/or efficient testing of the sample or a component thereof.

According to another embodiment, the sample sensor is a capacitive sample sensor and/or comprises a capacitor for monitoring or measuring the sample and/or is designed to gain information about the sample by measuring a capacity.

In particular, the sample sensor can be designed to measure or detect a temperature, a density, a transparency, a cloudiness, a color and/or a viscosity of the sample or a component thereof. In this way, a state of the sample can be easily determined or detected.

Preferably, the analysis device is designed to automatically place the sample sensor and/or the cartridge in an operating position upon or after insertion of the cartridge into the analysis device, in particular so that the sample in the receiving cavity can be monitored by the sample sensor in the operating position. This is conducive to an easy operation of the analysis device by a user. Further, errors in placing or positioning the sample sensor or in measuring the state of the sample can be avoided.

The analysis device preferably comprises a control apparatus for preferably automatically starting and/or controlling testing of the sample, in particular on the basis of signals received by the sample sensor. Thus, the sample sensor is preferably connected to the control apparatus or can be connected to the control apparatus, in particular electrically and/or by a data connection. Preferably, testing of the sample is started when or as soon as a sufficient or complete sedimentation or a separation of components of the sample are measured or detected by the sample sensor. This is conducive to a fast, reliable and/or efficient testing of the sample or a component thereof.

The analysis device preferably comprises a receiving unit for receiving, positioning and/or holding the cartridge and/or a connection unit for mechanically, electrically, thermally and/or fluidically connecting the cartridge. Preferably, the receiving unit and/or the connection unit comprise the sample sensor. It is preferred that the receiving unit can be moved relative to the connection unit in order to hold the cartridge in a clamped manner between said receiving unit and said connection unit and/or to connect the cartridge to the connection unit and/or to position the cartridge on said connection unit. This provides for a simple, robust and/or or cost-effective construction. A simple and/or reliable sequence is also made possible.

According to a further aspect which can also be realized independently, the present invention relates to a cartridge for receiving and/or testing the sample.

The cartridge preferably comprises a main body with a plurality of channels and cavities. Further, the cartridge preferably comprises a cover covering the channels and cavities.

Preferably, the cartridge comprises the sample sensor or a part thereof, in particular a reflector or reflecting part of the sample sensor.

As an alternative or in addition, the receiving cavity and/or cover of the cartridge is preferably at least partly and/or on one side provided with or covered by an additional or further cover or layer. The cover or the further/additional cover or layer is preferably made of an inorganic material or metal, at least in the area of the receiving cavity or in the part covering the receiving cavity. In particular, the cover or further/additional cover or layer is made of aluminum, at least in the area or part covering the receiving cavity.

Preferably, the cartridge comprises a receiving cavity for the sample and the analysis system comprises a sample sensor for monitoring the sample in the cartridge or receiving cavity. Preferably, the analysis device comprises the sample sensor. However, it is also possible that the cartridge comprises the sample sensor or at least parts thereof.

The receiving cavity preferably comprises an intermediate connection which is in particular provided in addition to an inlet and/or an outlet of the receiving cavity. The intermediate connection is preferably designed for discharging or removing a supernatant of the sample from the receiving cavity.

It is preferred that one or more preferably initially closed valves are assigned to the receiving cavity, in particular to the intermediate connection and/or the outlet. In this way, it can be easily achieved that the sample does not leave the receiving cavity before a sedimentation or separation of the sample is completed or sufficiently advanced. This is conducive to a fast, reliable and/or efficient testing of the sample or a component thereof.

According to a further aspect which can also be realized independently, the present invention relates to an analysis system for testing the sample.

The analysis system preferably comprises the analysis device and the cartridge for receiving or testing the sample.

According to a further aspect which can also be implemented independently, the present invention relates to a method for testing the sample with the analysis system. The sample is (in a first step) introduced in the receiving cavity of the cartridge.

Preferably, the sample in the receiving cavity is monitored by the sample sensor, wherein further processing of the sample or starting the test is based on signals of the sample sensor.

In particular, the sample is kept in the receiving cavity until a supernatant or sufficient or complete sedimentation or separation of the sample is measured or detected, in particular by means of the sample sensor. As an alternative or in addition, the outlet and/or the intermediate connection of the receiving cavity is kept closed until and/or only opened when or opened as soon as a supernatant or sufficient or complete sedimentation or separation of the sample is measured or detected, in particular by means of the sample sensor. This is conducive to a fast, reliable and/or efficient testing of the sample or a component thereof.

The term “analysis device” is preferably understood to mean an instrument which is in particular mobile and/or can be used on site, and/or which is designed to chemically, biologically and/or physically test and/or analyze a sample or a component thereof, preferably in and/or by means of a cartridge. In particular, the analysis device controls the pretreatment and/or testing of the sample in the cartridge. For this purpose, the analysis device can act on the cartridge, in particular such that the sample is conveyed, temperature-controlled and/or measured in the cartridge.

The term “cartridge” is preferably understood to mean a structural apparatus or unit designed to receive, to store, to physically, chemically and/or biologically treat and/or prepare and/or to measure a sample, preferably in order to make it possible to detect, identify or determine at least one analyte, in particular a protein and/or a nucleic-acid sequence, of the sample.

A cartridge within the meaning of the present invention preferably comprises a fluid system having a plurality of channels, cavities and/or valves for controlling the flow through the channels and/or cavities.

In particular, within the meaning of the present invention, a cartridge is designed to be at least substantially planar, flat and/or card-shaped, and in particular is designed as a (micro)fluidic card and/or is designed as a main body or container that can preferably be closed and/or said cartridge can be inserted and/or plugged into a proposed analysis device when it contains the sample.

The above-mentioned aspects and features of the present invention and the aspects and features of the present invention that will become apparent from the following description can in principle be implemented independently from one another, but also in any combination or order.

Other aspects, advantages, features and properties of the present invention will become apparent from the following description of a preferred embodiment with reference to the accompanying drawings.

### BRIEF DESCRIPTION OF THE DRAWINGS

FIG. 1 is a schematic view of a proposed analysis system comprising a proposed analysis device and a cartridge received in the analysis device;

FIG. 2 is a schematic view of the cartridge;

FIG. 3 is a schematic perspective front view of the cartridge;

FIG. 4 is a schematic perspective rear view of the cartridge;

FIG. 5 is a schematic perspective view of the analysis device when the housing is closed;

FIG. 6 is a schematic sectional view of the analysis device when the housing is open and the cartridge is received in part;

FIG. 7 is a schematic sectional view of the analysis device when the entire cartridge has been received and the housing is closed;

FIG. 8 is a schematic sectional view of the analysis device, showing the cartridge in a test position and showing unactuated valves;

FIG. 9 is a schematic sectional view of the analysis device, showing the cartridge in the test position and showing actuated valves; and

FIG. 10 is a schematic sectional view of a receiving cavity with a separated sample.

### DETAILED DESCRIPTION OF THE INVENTION

In the figures, which are only schematic and sometimes not to scale, the same reference signs are used for the same or similar parts and components, corresponding or comparable properties and advantages being achieved even if these are not repeatedly described.

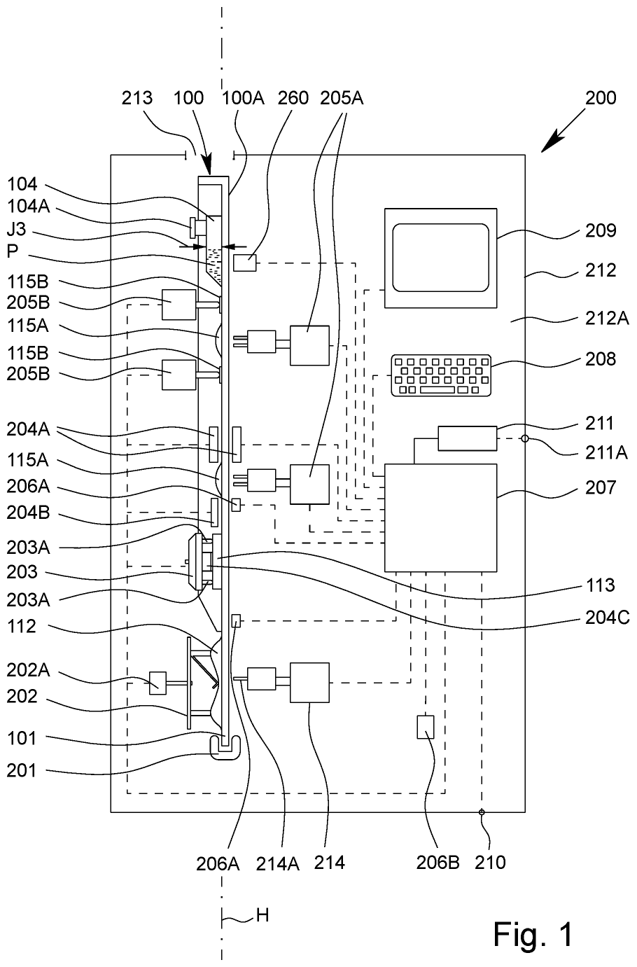

FIG. 1 is a highly schematic view of a proposed analysis system 1 and analysis device 200 for testing an in particular biological sample P, preferably by means of or in an apparatus or cartridge 100.

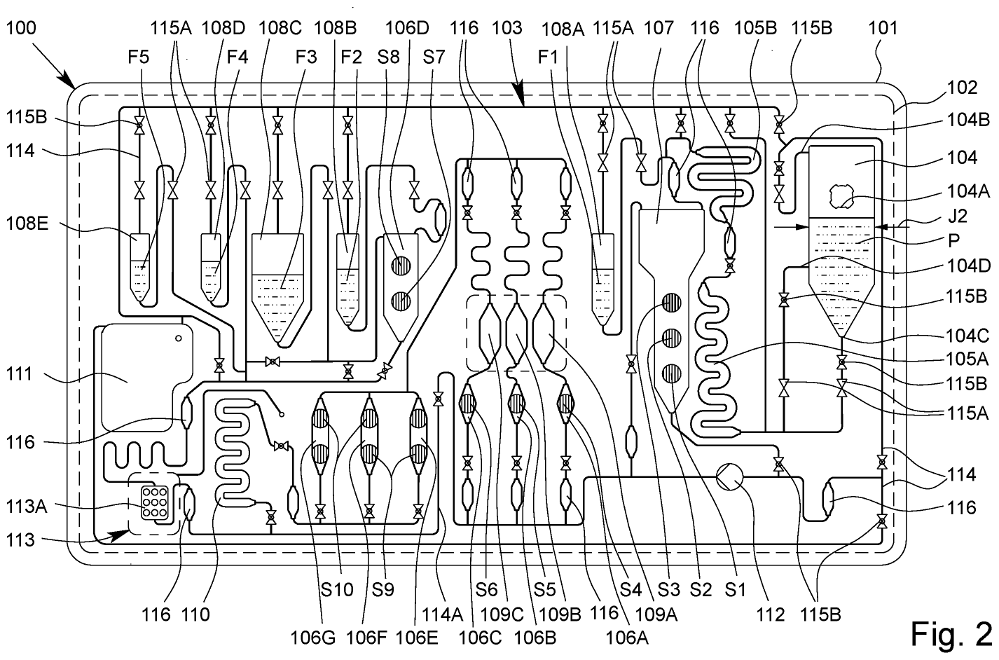

FIG. 2 is a schematic view of a preferred embodiment of the proposed apparatus or cartridge 100 for testing the sample P. The apparatus or cartridge 100 in particular forms a handheld unit, and in the following is merely referred to as a cartridge 100.

The term “sample” is preferably understood to mean the sample material to be tested, which is in particular taken from a human or animal. In particular, within the meaning of the present invention, a sample P is a fluid, such as saliva, blood, urine or another liquid, preferably from a human or animal, or a component thereof. The sample P may be derived from animals such as mammals including swine, horses, cattle, sheep, camelids and the like or from poultry such as chicken, turkey or other avians. Further, the sample P may be derived from food producing animals such as swine, cattle, sheep, fish etc. or from pet animals such as dogs, cats or equines.

Within the meaning of the present invention, a sample P may be pretreated or prepared if necessary, or may come directly from a human or animal or the like, for example. A food sample, environmental sample or another sample may optionally also be tested, in particular for environmental analytics, food safety and/or for detecting other substances, preferably natural substances, but also biological or chemical warfare agents, poisons or the like.

A sample P within the meaning of the present invention preferably contains one or more analytes, it preferably being possible for the analytes to be identified or detected, in particular qualitatively and/or quantitatively determined. Particularly preferably, within the meaning of the present invention, a sample P has target nucleic-acid sequences as the analytes, in particular target DNA sequences and/or target RNA sequences, and/or target proteins as the analytes, in particular target antigens and/or target antibodies. Particularly preferably, at least one disease and/or pathogen can be identified or detected in the sample P by qualitatively and/or quantitatively determining the analytes.

Preferably, the analysis system 1 or analysis device 200 controls the testing of the sample P in or on the cartridge 100 and/or is used to evaluate the testing and/or to collect, to process and/or to store measured values from the test.

By means of the proposed analysis system 1 or analysis device 200 and/or by means of the cartridge 100 and/or using the proposed method for testing the sample P, preferably an analyte of the sample P, in particular a (certain) nucleic-acid sequence or target nucleic-acid sequence and/or a (certain) protein or target protein, or particularly preferably a plurality of analytes of the sample P, can preferably be determined, detected or identified. Said analytes are in particular detected or identified and/or measured not only qualitatively, but particularly preferably also quantitatively.

Therefore, the sample P can in particular be tested for qualitatively or quantitatively determining at least one analyte, for example in order for it to be possible to detect or identify a disease and/or pathogen or to determine other values, which are important for diagnostics, for example.

Particularly preferably, a molecular-biological test is made possible by means of the analysis system 1 and/or analysis device 200 and/or by means of the cartridge 100.

Particularly preferably, a nucleic-acid assay for detecting or identifying a target nucleic-acid sequence, in particular a target DNA sequence and/or a target RNA sequence, and/or a protein assay for detecting or identifying a target protein, in particular a target antigen and/or target antibody, are made possible or are carried out.

The term “assay” is preferably understood to mean an in particular molecular-biological test for detecting or identifying at least one analyte in a sample P. In particular, at least one analyte in a sample P can be qualitatively or quantitatively detected or identified by means of an assay or by carrying out an assay. A plurality of method steps are preferably required to (fully) carry out an assay. Preferably, within the meaning of the present invention, when carrying out an assay, a sample P is pretreated with one or more reagents and the pretreated sample P is tested, in particular at least one analyte in the sample P being detected or identified. Within the meaning of the present invention, an assay is in particular an immunoassay or protein assay for detecting or identifying a target protein, in particular a target antigen and/or target antibody, and/or a nucleic-acid assay for detecting or identifying a target nucleic-acid sequence, in particular a target DNA sequence and/or target RNA sequence.

Preferably, the sample P or individual components of the sample P or analyte can be amplified if necessary, in particular by means of PCR, and tested, detected or identified in the analysis system 1 or analysis device 200 or in the cartridge 100, and/or for the purpose of carrying out the nucleic-acid assay. Preferably, amplification products of the analyte or analytes are thus produced.

In particular, the bonded analytes A or the amplification products thereof are electrochemically identified or detected both in the nucleic-acid assay and the protein assay.

In the following, further details are first given on a preferred construction of the cartridge 100, with features of the cartridge 100 preferably also directly representing features of the analysis system 1, in particular even without any further explicit explanation.

The cartridge 100 is preferably at least substantially planar, plate-shaped, flat and/or card-shaped.

The cartridge 100 preferably comprises an in particular at least substantially planar, flat, plate-shaped and/or card-shaped support or main body 101, the support or main body 101 in particular being made of and/or injection-molded from plastics material, particularly preferably polypropylene.

The cartridge 100 preferably comprises at least one film or cover 102 for covering the main body 101 and/or cavities and/or channels formed therein at least in part, in particular on the front 100A, and/or for forming valves or the like, as shown by dashed lines in FIG. 2.

Particularly preferably, the cover 102 completely covers the cavities and/or channels on the front 100A and/or on a flat side of the cartridge 100. In particular, the cover 102 covers all of the cavities and/or channels on the front 100A and/or on a flat side of the cartridge 100. The cartridge 100 and/or the main body 101 thereof, in particular together with the cover 102, preferably forms and/or comprises a fluidic system 103, referred to in the following as the fluid system 103.

The cartridge 100, the main body 101 and/or the fluid system 103 are preferably at least substantially vertically oriented in the operating position and/or during the test, in particular in the analysis device 200, as shown schematically in FIG. 1. In particular, the surface extension or main plane H of the cartridge 100 thus extends at least substantially vertically in the operating position.

The cartridge 100 and/or the fluid system 103 preferably comprises a plurality of cavities, in particular at least one receiving cavity 104, at least one metering cavity 105, at least one intermediate cavity 106, at least one mixing cavity 107, at least one storage cavity 108, at least one reaction cavity 109, at least one intermediate temperature-control cavity 110 and/or at least one collection cavity 111, the cavities preferably being fluidically interconnected by a plurality of channels.

Within the meaning of the present invention, channels are preferably elongate forms for conducting a fluid in a main flow direction or conveying direction, the forms preferably being closed transversely, in particular perpendicularly, to the main flow direction and/or longitudinal extension, preferably on all sides.

In particular, the main body 101 comprises elongate notches, recesses, depressions or the like, which are closed at the sides by the cover 102 and form channels within the meaning of the present invention.

Within the meaning of the present invention, cavities or chambers are preferably formed by recesses, depressions or the like in the cartridge 100 or main body 101, which are closed or covered by the cover 102, in particular at the sides. The volume or space enclosed by each cavity is preferably fluidically linked, in particular to the fluid system 103, by means of the channels.

In particular, within the meaning of the present invention, a cavity comprises at least two openings for the inflow and/or outflow of fluids.

Within the meaning of the present invention, cavities preferably have a larger diameter and/or flow cross section and/or a larger volume than channels, preferably by at least a factor of 2, 3 or 4. In principle, however, cavities may in some cases also be elongate, in a similar manner to channels.

Preferably, within the meaning of the present invention, a cavity comprises at least two openings for the inflow and/or outflow of fluids and/or comprises an inlet and an outlet, in particular such that said fluid can flow through the cavities from the inlet to the outlet.

Preferably, several or all of the cavities are vertically oriented and/or are oriented such that fluid can flow through the cavities at least substantially vertically in the normal operating position of the cartridge 100.

Particularly preferably, several or all of the cavities, in particular the receiving cavity 104, the intermediate cavity/cavities 106, the mixing cavity 107, the storage cavity/cavities 108 and/or the reaction cavity/cavities 109, are elongate, the longitudinal extension of the cavities preferably extending at least substantially vertically, and/or in parallel with gravity in the normal operating position of the cartridge 100.

Preferably, the inlet of several or all of the cavities is at the top in the normal operating position of the cartridge 100 and the outlet of several or all of the cavities is at the bottom in the normal operating position of the cartridge 100, in particular such that fluid can flow through or drain from some or all of the cavities, in particular the storage cavity/cavities 108, from the top to the bottom in the normal operating position and/or a fluid located in the cavities, in particular the storage cavity/cavities 108, can be removed and/or pumped out at the bottom. In this way, bubble formation and/or foaming of the fluids located in the cavities can be prevented. In particular, this prevents a gas, in particular air, from being conveyed out of the cavities.

The analysis system 1, in particular the cartridge 100 and/or the fluid system 103, also preferably comprises at least one pump apparatus 112 and/or at least one sensor arrangement or sensor apparatus 113.

In the example shown, the cartridge 100 or the fluid system 103 preferably comprises two metering cavities 105A and 105B, a plurality of intermediate cavities 106A to 106G, a plurality of storage cavities 108A to 108E and/or a plurality of reaction cavities 109, which can preferably be loaded separately from one another, in particular a first reaction cavity 109A, a second reaction cavity 109B and an optional third reaction cavity 109C, as can be seen in FIG. 2.

The metering cavities 105 are preferably designed to receive, to temporarily store and/or to meter the sample P, and/or to pass on said sample in a metered manner Particularly preferably, the metering cavities 105 have a diameter which is larger than that of the (adjacent) channels.

In the initial state of the cartridge 100 or when at the factory or in storage, the storage cavities 108 are preferably filled at least in part, in particular with a liquid such as a reagent, solvent or wash buffer.

The collection cavity 111 is preferably designed to receive larger quantities of fluids that are in particular used for the test, such as reagents, sample residues or the like. Preferably, in the initial state or when at the factory, the collection cavity 111 is empty or filled with gas, in particular air. The volume of the collection cavity 111 corresponds to or exceeds preferably the (cumulative) volume of the storage cavity/cavities 108 or the liquid content thereof and/or the volume of the receiving cavity 104 or the sample P received.

The reaction cavity/cavities 109 is/are preferably designed to allow a substance located in the reaction cavity 109 to react when an assay is being carried out, for example by being linked or coupled to apparatuses or modules of the analysis device 200.

The reaction cavity/cavities 109 is/are used in particular to carry out an amplification reaction, in particular PCR, or several, preferably different, amplification reactions, in particular PCRs. It is preferable to carry out several, preferably different, PCRs, i.e., PCRs having different primer combinations or primer pairs, in parallel and/or separately and/or in different reaction cavities 109.

To carry out the nucleic-acid assay, preferably target nucleic-acid sequences, as analytes of the sample P, are amplified in the reaction cavity/cavities 109 by means of an amplification reaction, in particular in order to produce amplification products for the subsequent detection in the sensor arrangement or sensor apparatus 113.

Within the meaning of the present invention, amplification reactions are in particular molecular-biological reactions in which an analyte, in particular a target nucleic-acid sequence, is amplified/copied and/or in which amplification products, in particular nucleic-acid products, of an analyte are produced. Particularly preferably, PCRs are amplification reactions within the meaning of the present invention.

“PCR” stands for polymerase chain reaction and is a molecular-biological method by means of which certain analytes, in particular portions of RNA or RNA sequences or DNA or DNA sequences, of a sample P are amplified, preferably in several cycles, using polymerases or enzymes, in particular in order to then test and/or detect the amplification products or nucleic-acid products. If RNA is intended to be tested and/or amplified, before the PCR is carried out, a cDNA is produced starting from the RNA, in particular using reverse transcriptase. The cDNA is used as a template for the subsequent PCR.

Preferably, during a PCR, a sample P is first denatured by the addition of heat in order to separate the strands of DNA or cDNA. Preferably, primers or nucleotides are then deposited on the individual separated strands of DNA or cDNA, and a desired DNA or cDNA sequence is replicated by means of polymerase and/or the missing strand is replaced by means of polymerase. This process is preferably repeated in a plurality of cycles until the desired quantity of the DNA or cDNA sequence is available.

For the PCR, marker primers are preferably used, i.e., primers which (additionally) produce a marker or a label, in particular biotin, on the amplified analyte or amplification product. This allows or facilitates detection. Preferably, the primers used are biotinylated and/or comprise or form in particular covalently bonded biotin as the label.

The amplification products, target nucleic-acid sequences and/or other portions of the sample P produced in the one or more reaction cavities 109 can be conducted or fed to the connected sensor arrangement or sensor apparatus 113, in particular by means of the pump apparatus 112.

The sensor arrangement or sensor apparatus 113 is used in particular for detecting, particularly preferably qualitatively and/or quantitatively determining, the analyte or analytes of the sample P, in this case particularly preferably the target nucleic-acid sequences and/or target proteins as the analytes. Alternatively or additionally, however, other values may also be collected or determined.

The sensor apparatus 113 preferably comprises a sensor array 113A in order for it to be possible to determine or detect in particular a plurality of analytes. Preferably, electrochemical detection is carried out.

Preferably, the sensor arrangement or sensor apparatus 113 is provided with capture molecules for bonding the analytes A. In particular, the sensor arrangement or sensor apparatus 113 is designed to electrochemically detect analytes A bonded to the capture molecules.

The sensor arrangement or sensor apparatus 113 preferably comprises (precisely) one sensor array 113A comprising a plurality of sensor fields and/or electrodes, the sensor fields and/or electrodes each being in particular provided with capture molecules.

Within the meaning of the present invention, capture molecules are in particular nucleic-acid sequences, in particular DNA sequences and/or RNA sequences, and/or proteins, in particular antigens and/or antibodies. In particular, the capture molecules M are designed to bond and/or immobilize corresponding analytes A of the sample P.

Within the meaning of the present invention, capture molecules are in particular applied to, fixed to and/or immobilized on a sensor array 113A, in particular the sensor fields and/or electrodes of the sensor array 113A, in a process known as spotting.

Preferably, the sensor array 113A, the sensor fields and/or electrodes are surface-treated or coated, in particular with thiols, in order to immobilize the capture molecules, in particular in order to make it possible to bond the capture molecules to the electrodes.

The cartridge 100, the main body 101 and/or the fluid system 103 preferably comprise a plurality of channels 114 and/or valves 115, as shown in FIG. 2.

By means of the channels 114 and/or valves 115, the cavities 104 to 111, the pump apparatus 112 and/or the sensor arrangement or sensor apparatus 113 can be temporarily and/or permanently fluidically interconnected, in particular to form a fluidic circuit, and/or fluidically separated from one another, as required and/or optionally or selectively, in particular such that they are controlled by the analysis system 1 or the analysis device 200.

The pump apparatus 112 is preferably configured for pumping the sample P out of the receiving cavity and/or for transporting the sample P through the cartridge 100, in particular through the channels 114 and/or cavities 104 to 111.

The cavities 104 to 111 are preferably each fluidically linked or interconnected by a plurality of channels 114. Particularly preferably, each cavity is linked or connected by at least two associated channels 114, in order to make it possible for fluid to fill, flow through and/or drain from the respective cavities as required.

The fluid transport or the fluid system 103 is preferably not based on capillary forces, or is not exclusively based on said forces, but in particular is essentially based on the effects of gravity and/or pumping forces and/or compressive forces and/or suction forces that arise, which are particularly preferably generated by the pump or pump apparatus 112. In this case, the flows of fluid or the fluid transport and the metering are controlled by accordingly opening and closing the valves 115 and/or by accordingly operating the pump or pump apparatus 112, in particular by means of a pump drive 202 of the analysis device 200.

Preferably, each of the cavities 104 to 110 has an inlet at the top and an outlet at the bottom in the operating position. Therefore, if required, only liquid from the respective cavities can be removed via the outlet.

In the operating position, the liquids from the respective cavities are preferably removed, in particular drawn out, via the outlet that is at the bottom in each case, it preferably being possible for gas or air to flow and/or be pumped into the respective cavities via the inlet that is in particular at the top. In particular, relevant vacuums in the cavities can thus be prevented or at least minimized when conveying the liquids.

In particular, the cavities, particularly preferably the storage cavity/cavities 108, the mixing cavity 107 and/or the receiving cavity 104, are each dimensioned and/or oriented in the normal operating position such that, when said cavities are filled with liquid, bubbles of gas or air that may potentially form rise upwards in the operating position, such that the liquid collects above the outlet without bubbles. However, other solutions are also possible here.

The receiving cavity 104 is preferably arranged upstream of the sensor apparatus 113 and/or all other cavities 105 to 111, in particular the metering cavity/cavities 105(A,B), the intermediate cavity/cavities 106(A-G), the mixing cavity 107, the storage cavity/cavities 108(A-E), the reaction cavity/cavities 109(A-C), the intermediate temperature-control cavity 110 and/or the collection cavity 111. In other the words, the sensor apparatus 113 and/or the cavities 105 to 111 are arranged downstream of the receiving cavity 104.

The receiving cavity 104 preferably comprises a connection 104A for introducing the sample P. In particular, the sample P may for example be introduced into the receiving cavity 104 and/or cartridge 100 via the connection 104A by means of a pipette, syringe or other instrument.

Preferably, the connection 104A directly connects the receiving cavity 104 to an outside of the cartridge 100 so that the sample can be directly introduced into the receiving cavity 104. In other words, it is preferred that no further cavities and/or channels are arranged between the connection 104A and the outside of the cartridge 100 and/or between the receiving cavity 104 and the connection 104A.

The receiving cavity 104 preferably comprises an inlet 104B, an outlet 104C and an optional intermediate connection 104D, it preferably being possible for the sample P or a portion thereof to be removed and/or conveyed further via the outlet 104C and/or the optional intermediate connection 104D. Gas, air or another fluid can flow in and/or be pumped in via the inlet 104B, as already explained.

The connection 104 is preferably provided in addition to the inlet 104B, outlet 104C and/or intermediate connection 104D.

Preferably, the sample P or a portion thereof can be removed, optionally and/or depending on the assay to be carried out, via the outlet 104C or the optional intermediate connection 104D of the receiving cavity 104. In particular, a supernatant of the sample P, such as blood plasma or blood serum, can be discharged or removed via the optional intermediate connection 104D, in particular for carrying out the protein assay.

Preferably, at least one valve 115 is assigned to each cavity, the pump apparatus 112 and/or the sensor apparatus 113 and/or is arranged upstream of the respective inlets and/or downstream of the respective outlets.

Preferably, the cavities 104 to 111 or sequences of cavities 104 to 111, through which fluid flows in series or in succession for example, can be selectively released and/or fluid can selectively flow therethrough by the assigned valves 115 being actuated, and/or said cavities can be fluidically connected to the fluid system 103, in particular a fluidic, preferably closed circuit of the fluid system 103, and/or to other cavities.

In particular, the valves 115 are formed by the main body 101 and the film or cover 102 and/or are formed therewith and/or are formed in another manner, for example by or having additional layers, depressions or the like.

Particularly preferably, one or more valves 115A are provided which are preferably tightly closed initially or when in storage, particularly preferably in order to seal liquids or liquid reagents F, located in the storage cavities 108, and/or the fluid system 103 from the open receiving cavity 104 in a storage-stable manner

Preferably, an initially closed valve 115A is arranged upstream and downstream of each storage cavity 108. These valves are preferably only opened, in particular automatically, when the cartridge 100 is actually being used and/or during or after (first) inserting the cartridge 100 into the analysis device 200 and/or for carrying out the assay.

A plurality of valves 115A, in particular three valves in this case, are preferably assigned to the receiving cavity 104, in particular if the intermediate connection 104D is provided in addition to the inlet 104B and the outlet 104C. Depending on the use, in addition to the valve 115A on the inlet 104B, then preferably only the valve 115A either at the outlet 104C or at the intermediate connection 104D is opened.

The valves 115A assigned to the receiving cavity 104 seal the fluid system 103 and/or the cartridge 100 in particular fluidically and/or in a gas-tight manner, preferably until the sample P is introduced and/or the receiving cavity 104 or the connection 104A of the receiving cavity 104 is closed.

As an alternative or in addition to the valves 115A (which are initially closed), one or more valves 115B are preferably provided which are not closed in a storage-stable manner and/or which are open initially or in an inoperative position, in an initial state or when the cartridge 100 is not inserted into the analysis device 200, and/or which can be closed by actuation. These valves 115B are used in particular to control the flows of fluid during the test.

The cartridge 100 is preferably designed as a microfluidic card and/or the fluid system 103 is preferably designed as a microfluidic system. In the present invention, the term “microfluidic” is preferably understood to mean that the respective volumes of individual cavities, some of the cavities or all of the cavities 104 to 111 and/or channels 114 are, separately or cumulatively, less than 5 ml or 2 ml, particularly preferably less than 1 ml or 800 μl, in particular less than 600 μl or 300 μl, more particularly preferably less than 200 μl or 100 μl.

Particularly preferably, a sample P having a maximum volume of 5 ml, 2 ml or 1 ml can be introduced into the cartridge 100 and/or the fluid system 103, in particular the receiving cavity 104.

Reagents and liquids which are preferably introduced or provided before the test in liquid form as liquids or liquid reagents F and/or in dry form as dry reagents S are required for testing the sample P, as shown in the schematic view according to FIG. 2 by reference signs F1 to F5 and S1 to S10.

Furthermore, other liquids F, in particular in the form of a wash buffer, solvent for dry reagents S and/or a substrate, for example in order to form detection molecules D and/or a redox system, are also preferably required for the test, the detection process and/or for other purposes, and are in particular provided in the cartridge 100, i.e., are likewise introduced before use, in particular before delivery. At some points in the following, a distinction is not made between liquid reagents and other liquids, and therefore the respective explanations are accordingly also mutually applicable.

The analysis system 1 or the cartridge 100 preferably contains all the reagents and liquids required for pretreating the sample P and/or for carrying out the test or assay, in particular for carrying out one or more amplification reactions or PCRs, and therefore, particularly preferably, it is only necessary to receive the optionally pretreated sample P.

The cartridge 100 or the fluid system 103 preferably comprises a bypass 114A that can optionally be used, in order for it to be possible, if necessary, to conduct or convey the sample P or components thereof past the reaction cavities 109 and/or, by bypassing the optional intermediate temperature-control cavity 110, also directly to the sensor arrangement or sensor apparatus 113.

Preferably, the bypass 114A is used when carrying out the protein assay, in particular in order to feed the sample P or a portion thereof directly from the mixing cavity 107 to the sensor arrangement or sensor apparatus 113, and/or to conduct said sample or portion past the reaction cavities 109 and/or the intermediate temperature-control cavity 110.

The cartridge 100 or the fluid system 103 and/or the channels 114 preferably comprise sensor portions 116 or other apparatuses for detecting liquid fronts and/or flows of fluid.

It is noted that various components, such as the channels 114, the valves 115, in particular the valves 115A that are initially closed and the valves 115B that are initially open, and the sensor portions 116 in FIG. 2 are, for reasons of clarity, only labeled in some cases, but the same symbols are used in FIG. 2 for each of these components.

The collection cavity 111 is preferably used for receiving excess or used reagents and liquids and volumes or portions of the sample, and/or for providing gas or air in order to empty individual cavities and/or channels. In the initial state, the collection cavity 111 is preferably filled solely with gas, in particular air.

In particular, the collection cavity 111 can optionally be connected to individual cavities and channels 114 or other apparatuses fluidically and/or so as to form a fluidic circuit, in order to remove reagents and liquids from said cavities, channels or other apparatuses and/or to replace said reagents and liquids with gas or air in particular from the collection cavity 111. The collection cavity 111 is preferably given appropriate (large) dimensions.

Once the sample P has been introduced into the receiving cavity 104 and the connection 104A has been closed, the cartridge 100 can be inserted into and/or received in the proposed analysis device 200 in order to test the sample P, as shown in FIG. 1.

FIG. 1 shows the analysis system 1 in a ready-to-use state for carrying out a test or assay on the sample P received in the cartridge 100. In this state, the cartridge 100 is therefore linked to, received by and/or inserted into the analysis device 200.

In the following, some features and aspects of the analysis device 200 are first explained in greater detail, in particular on the basis of FIG. 1. The features and aspects relating to said device are preferably also directly features and aspects of the proposed analysis system 1, in particular even without any further explicit explanation.

The analysis system 1 or analysis device 200 preferably comprises an in particular slot-like mount or receptacle 201 for preferably vertically mounting and/or receiving the cartridge 100.

Preferably, the cartridge 100 is fluidically, in particular hydraulically, separated or isolated from the analysis device 200. In particular, the cartridge 100 forms a preferably independent and in particular closed or sealed fluidic or hydraulic system 103 for the sample P and the reagents and other liquids. In this way, the analysis device 200 does not come into direct contact with the sample P and in particular can be reused for another test without having to be disinfected and/or cleaned first.

It is however provided that the analysis device 200 is or can be connected or coupled mechanically, electrically, thermally and/or pneumatically to the cartridge 100, in particular on one of the flat sides of the cartridge 100 and/or laterally. In particular, after receiving the cartridge 100, the analysis device 200 mechanically, thermally and/or pneumatically acts on the cartridge 100 on at least one of the flat sides of the cartridge 100 and/or laterally.

In particular, the analysis device 200 is designed to have a mechanical effect, in particular for actuating the pump apparatus 112 and/or the valves 115, and/or to have a thermal effect, in particular for temperature-controlling the reaction cavity/cavities 109 and/or the intermediate temperature-control cavity 110.

In addition, the analysis device 200 can preferably be pneumatically connected to the cartridge 100, in particular in order to actuate individual apparatuses, and/or can be electrically connected to the cartridge 100, in particular in order to collect and/or transmit measured values, for example from the sensor apparatus 113 and/or sensor portions 116.

The analysis system 1 or analysis device 200 preferably comprises a pump drive 202, the pump drive 202 in particular being designed for mechanically actuating the pump apparatus 112.

The analysis system 1 or analysis device 200 preferably comprises a connection apparatus 203 for in particular electrically and/or thermally connecting the cartridge 100 and/or the sensor arrangement or sensor apparatus 113.

As shown in FIG. 1, the connection apparatus 203 preferably comprises a plurality of electrical contact elements 203A, the cartridge 100, in particular the sensor arrangement or sensor apparatus 113, preferably being electrically connected or connectable to the analysis device 200 by the contact elements 203A. The contact elements 203A are preferably contact springs; however, they may also be spring-loaded connection pins or the like.

The analysis system 1 or analysis device 200 preferably comprises one or more temperature-control apparatuses 204 for temperature-controlling the cartridge 100 and/or having a thermal effect on the cartridge 100, in particular for heating and/or cooling, the temperature-control apparatus(es) 204 (each) preferably comprising or being formed by a heating resistor or a Peltier element.

Preferably, individual temperature-control apparatuses 204, some of these apparatuses or all of these apparatuses can be positioned against the cartridge 100, the main body 101, the cover 102, the sensor arrangement, sensor apparatus 113 and/or individual cavities and/or can be thermally coupled thereto and/or can be integrated therein and/or can be operated or controlled in particular electrically by the analysis device 200. In the example shown, in particular the temperature-control apparatuses 204A, 204B and/or 204C are provided.

The analysis device 200 preferably comprises one or more actuators 205 for actuating the valves 115. Particularly preferably, different (types or groups of) actuators 205A and 205B are provided which are assigned to the different (types or groups of) valves 115A and 115B for actuating each of said valves, respectively.

The analysis system 1 or analysis device 200 preferably comprises one or more sensors 206. In particular, sensors 206A are assigned to the sensor portions 116 and/or are designed or intended to detect liquid fronts and/or flows of fluid in the fluid system 103.

Particularly preferably, the sensors 206A are designed to measure or detect, in particular in a contact-free manner, for example optically and/or capacitively, a liquid front, flow of fluid and/or the presence, the speed, the mass flow rate/volume flow rate, the temperature and/or another value of a fluid in a channel and/or a cavity, in particular in a respectively assigned sensor portion 116, which is in particular formed by a planar and/or widened channel portion of the fluid system 103.

Alternatively or additionally, the analysis device 200 preferably comprises (other or additional) sensors 206B for detecting the ambient temperature, internal temperature, atmospheric humidity, position, and/or alignment, for example by means of a GPS sensor, and/or the orientation and/or inclination of the analysis device 200 and/or the cartridge 100.

Particularly preferably, the analysis device 200 comprises a sensor 206B for detecting the horizontal and/or vertical orientation of the cartridge 100 and/or analysis device 200, the sensor 206B preferably being designed as a tilt sensor or inclinometer. However, other solutions are also possible here, in particular those in which the analysis device 200 comprises a spirit level or level indicator in order to display the horizontal and/or vertical orientation of the cartridge 100 and/or analysis device 200.

The analysis system 1 or analysis device 200 preferably comprises a control apparatus 207, in particular comprising an internal clock or time base for controlling the sequence of a test or assay and/or for collecting, evaluating and/or outputting or providing measured values in particular from the sensor apparatus 113, and/or from test results and/or other data or values.

The control apparatus 207 preferably controls or feedback controls the pump drive 202, the temperature-control apparatuses 204 and/or actuators 205, in particular taking into account or depending on the desired test and/or measured values from the sensor arrangement or sensor apparatus 113 and/or sensors 206.

The flows of fluid are controlled in particular by accordingly activating the pump or pump apparatus 112 and actuating the valves 115.

Optionally, the analysis system 1 or analysis device 200 comprises an input apparatus 208, such as a keyboard, a touch screen or the like, and/or a display apparatus 209, such as a screen.

The analysis system 1 or analysis device 200 preferably comprises at least one interface 210, for example for controlling, for communicating and/or for outputting measured data or test results and/or for linking to other devices, such as a printer, an external power supply or the like. This may in particular be a wired or wireless interface 210.

The analysis system 1 or analysis device 200 preferably comprises a power supply 211 for providing electrical power, preferably a battery or an accumulator, which is in particular integrated and/or externally connected or connectable.

Preferably, an integrated accumulator is provided as a power supply 211 and is (re)charged by an external charging device (not shown) via a connection 211A and/or is interchangeable.

The analysis system 1 or analysis device 200 preferably comprises a housing 212, all the components and/or some or all of the apparatuses preferably being integrated in the housing 212. Particularly preferably, the cartridge 100 can be inserted or slid into the housing 212 or the mount 201, and/or can be received by the analysis device 200 or the mount 201, through an opening 213 which can in particular be closed, such as a slot or the like.

The analysis system 1 or analysis device 200 is preferably portable or mobile. Preferably, the analysis device 200 weighs less than 25 kg or 20 kg, particularly preferably less than 15 kg or 10 kg, in particular less than 9 kg or 6 kg.

As already explained, the analysis device 200 can preferably be fluidically and/or pneumatically linked or connected to the cartridge 100, in particular to the sensor arrangement or sensor apparatus 113 and/or to the pump apparatus 112, preferably by means of one or more connections 129.

Particularly preferably, the analysis device 200 is designed to supply the cartridge 100, in particular the sensor arrangement or sensor apparatus 113 and/or the pump apparatus 112, with a working medium, in particular gas or air.

Preferably, the working medium can be compressed and/or pressurized in the analysis device 200 or by means of the analysis device 200.

Preferably, the analysis device 200 comprises a pressurized gas supply 214, in particular a pressure generator and/or compressor, preferably in order to compress, condense and/or pressurize the working medium.

The pressurized gas supply 214 is preferably integrated in the analysis device 200 or the housing 212 and/or can be controlled or feedback controlled by means of the control apparatus 207.

Preferably, the pressurized gas supply 214 is electrically operated or can be operated by electrical power. In particular, the pressurized gas supply 214 can be supplied with electrical power by means of the power supply 211.

At the start of the proposed method, a sample P having at least one analyte, preferably a fluid or a liquid from the human or animal body, in particular blood, saliva or urine, is preferably first introduced into the receiving cavity 104 via the connection 104A, it being possible for the sample P to be pretreated, in particular filtered.

Once the sample P has been received, the receiving cavity 104 and/or the connection 104A thereof is fluidically closed, in particular in a liquid-tight and/or gas-tight manner.

Preferably, the cartridge 100 together with the sample P is then linked to the analysis device 200, in particular is inserted or slid at least in part into the analysis device 200 or the mount 201 or opening 213, particularly preferably from the top.

Particularly preferably, the cartridge 100 is received at least in part, at least substantially vertically, by the analysis device 200.

Preferably, the in particular vertical and/or horizontal orientation of the cartridge 100 and/or the analysis device 200 is measured, in particular electronically and/or by means of the sensor 206B, preferably before the test starts.

In particular, the in particular vertical and/or horizontal orientation of the cartridge 100 or the analysis device 200 is measured, in particular by means of the sensor 206B, immediately after the analysis device 200 is switched on and/or after the cartridge 100 is received. In particular, it is measured or established whether the main plane of extension H of the cartridge 100 extends vertically in the analysis device 200 and/or whether the analysis device 200 is oriented horizontally and/or positioned so as to be flat and/or is not tilted and/or not inclined.

Preferably, the measured orientation of the cartridge 100 and/or the analysis device 200 is displayed to a user, preferably by the display apparatus 209.

Preferably, the test is blocked or prevented, in particular the test is blocked or prevented from starting, particularly preferably electronically, if the orientation of the cartridge 100 is inclined or not vertical and/or if the orientation of the analysis device 200 is tilted or not horizontal. More particularly preferably, the sample P can only be tested when the cartridge 100 is at least essentially oriented vertically and/or when the analysis device 200 is at least essentially oriented horizontally.

If the cartridge 100 or the analysis device 200 is oriented so as to be inclined or tilted and/or is not oriented as desired, the orientation of the analysis device 200 and thus of the cartridge 100 is adapted.

Preferably, it is displayed, in particular by means of the display apparatus 209, when the correct or vertical orientation is set. The testing of the sample P can then start.

Preferably, the sample P or a part or supernatant of the sample P is removed from the receiving cavity 104 at the bottom or via the outlet 104C, preferably for carrying out the nucleic-acid assay, and/or centrally or via the intermediate connection 104D, in particular for carrying out the protein assay, and is preferably fed to the mixing cavity 107 in a metered manner

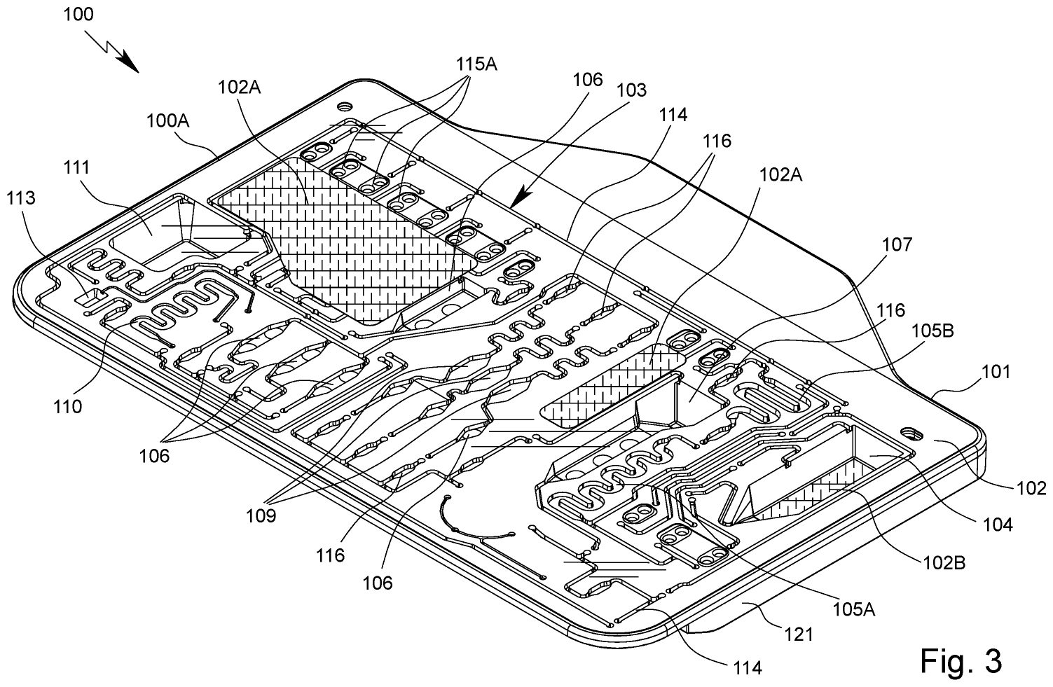

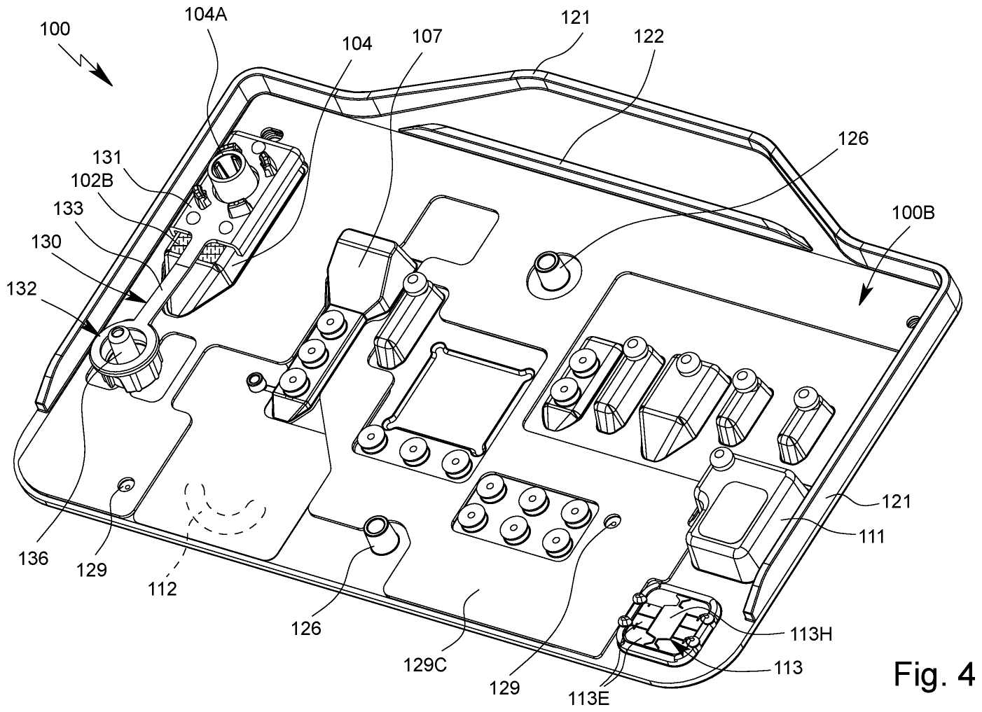

FIG. 3 is a perspective front view of the cartridge 100, i.e., of the front 100A thereof, and FIG. 4 is a perspective rear view of the cartridge 100, i.e., of the back 100B thereof.

The cover 102 is preferably made of plastics, in particular polypropylene and/or the same or at least essentially similar material as the main body 101.

The cover 102 is preferably produced from or additionally covered—partially or completely—by a different material, such as an inorganic material, in particular metal, particularly preferably aluminum, preferably in the region of at least one storage cavity 108 and alternatively or additionally other areas or cavities, such as the receiving cavity 104. This is preferably achieved by applying or adhesively bonding a piece of material or film sheet, consisting of or produced from the corresponding material, as an additional cover or layer 102A in the respective region(s). This is shown schematically in FIG. 3 for the storage cavities 108.

The other cavities, such as the receiving cavity 104, and/or the main body 101 can be covered in the same way.

The additional cover/layer 102A is preferably embodied as a (thin) foil or film.

In the example shown, for example an additional cover/layer 102A is assigned, in the region to the right of the center, to just one storage cavity, in this case the storage cavity 108A, in order to cover said storage cavity. On the left-hand side in FIG. 3, a larger piece of material, as the additional cover/layer 102A, preferably covers the entirety of a plurality of storage cavities 108, in this case the storage cavities 108B-108E.

The additional cover/layer 102A thus preferably does not cover the cover 102 completely, but only in part, in particular only in the region of one or more storage cavities 108 and/or the receiving cavity 104.

The additional cover/layer 102A is in each case preferably connected and/or adhesively bonded, over its entire surface, to the cover 102 located therebelow.

In principle, it is also possible to apply the additional cover/layer 102A in another manner, for example by coating and/or by lamination, adhesion or the like.

Accordingly, significantly improved storage stability of the liquid reagents F located in the storage cavities 108 can be achieved in a simple manner by applying the additional cover/layer 102A.

Further, the additional cover/layer 102A preferably reduces or prevents diffusion of substances stored or located within the storage cavities 108 and/or the receiving cavity 104. Further, the additional cover/layer 102A preferably improves thermal insulation and/or stability, i.e., in particular a constant or only little-varying temperature in the storage cavities 108 and/or the receiving cavity 104.

In the example shown, the additional cover/layer 102A is applied and/or adhesively bonded after the (continuous) cover 102 has been applied. The additional cover/layer 102A is therefore arranged on the side of the cover 102 remote or opposite from the main body 101.

The additional cover/layer 102A can alternatively also be applied first to the main body 101 and then covered by the continuous cover 102. This results in comparable advantages.

The additional cover can preferably be removed, in particular peeled off, from the cartridge 100 or cover 102 before inserting the cartridge 100 in the analysis device 200 and/or before testing.

As an alternative or in addition to the additional cover/layer 102A on the front 100A of the cartridge 100, at least the receiving cavity 104 can comprise or be provided with a further cover or layer 102B.

Preferably, the further cover/layer 102B is positioned on or in the main body 101 and/or covers the back 100B of the cartridge 100 or receiving cavity 104.

The further cover/layer 102B is preferably arranged on the outside of the receiving cavity 104, as indicated in FIG. 4. As an alternative or in addition, it is possible to arrange the further cover/layer 102B inside the receiving cavity 104A, as indicated in FIG. 3.

The further cover/layer 102B is preferably attached, in particular adhesively bonded or laminated, to the main body 101, in particular to a portion or area of the main body 101 forming the receiving cavity 104.

The additional or further cover/layer 102A, 102B is preferably made of the same material as the additional cover/layer 102A and/or a different material than the main body 101, such as an inorganic material or metal, in particular aluminum.

Thus, an aspect of the present invention which can be realized independently is that the receiving cavity 104 is at least partly and/or on one sided provided with or covered by the additional or further cover/layer 102A, 102B.

The further cover/layer 102B preferably facilitates and/or enhances monitoring of the sample P in the receiving cavity 104 by a sample sensor 206I which is described later.

The cartridge 100 or the main body 101 preferably comprises at least one positioning portion 126, in particular two positioning portions 126 in the example shown, for mounting and/or positioning the cartridge 100 in a defined manner, in particular in the analysis device 200 while a sample P is being tested, as shown in FIG. 4.

The positioning portion 126 is in particular integrally molded on or formed in one piece with the main body 101.

The positioning portion 126 preferably projects from a flat side, in this case the back 100B, or the plate plane of the cartridge 100 or main body 101.

The positioning portion 126 is in particular cylindrical or hollow cylindrical and/or conical, preferably on the inside and/or outside.

The outside of the positioning portion 126 preferably tapers towards the free end or is conical. This is conducive to simple production and/or centering of the cartridge 100 in the analysis device 200.

The inside of the positioning portion 126 is preferably conical or widens towards the free end. This is conducive to simple production and/or centering of the cartridge 100 in the analysis device 200.

The two positioning portions 126 are preferably arranged in a line that is parallel to a side of the cartridge 100, in particular in a central line that is transverse to a longitudinal side of the cartridge 100.

In particular, in the view according to FIG. 4, one positioning portion 126 is arranged in the region of the lower longitudinal side of the cartridge 100. The other positioning portion 126 is arranged in particular in the vicinity of an optional reinforcing rib 122.

The connection 104A of the receiving cavity 104 can be closed after the sample P has been received. The cartridge 100 preferably comprises a closure element 130 for this purpose.

In particular, the connection 104A can be closed in a liquid-tight and particularly preferably also gas-tight manner by the closure element 130. In particular, a closed fluid circuit can thus be formed, with the receiving cavity 104 being included. In particular, once the assigned valves 115A at the inlet 104B, outlet 104C and/or intermediate connection 104D have been opened, the receiving cavity 104 thus forms part of the fluid system 103 of the cartridge 100, wherein the fluid system is preferably closed or can be closed by the closure element 130.

The closure element 130 or the closure part 132 thereof closes the receiving cavity 104 or the connection 104A thereof preferably in a permanent manner, i.e., it preferably cannot be released again. The connection 104A therefore preferably cannot be reopened after it has been closed.

In the example shown, the closure element 130 preferably comprises a base part 131 and the closure part 132, the closure part 132 being movably and/or pivotally connected to the base part 131 in particular by means of a connecting part 133 that is preferably formed bar-like in this case.

Preferably, the base part 131, the connecting part 133 and the closure part 132 are formed in one piece, in particular formed as an injection-molded part and/or produced from plastics material.

Preferably, the receiving cavity 104 is filled with the sample P when the plate plane or main plane H of the cartridge 100 is oriented at least substantially horizontally and, after the connection 104A has been closed, the test is carried out or can be carried out on the received sample P, in this case in particular in the analysis device 200, when the plane H of the cartridge 100 is oriented at least substantially vertically. This at least substantially vertical orientation is therefore the preferred operating position of the cartridge 100 during the test.

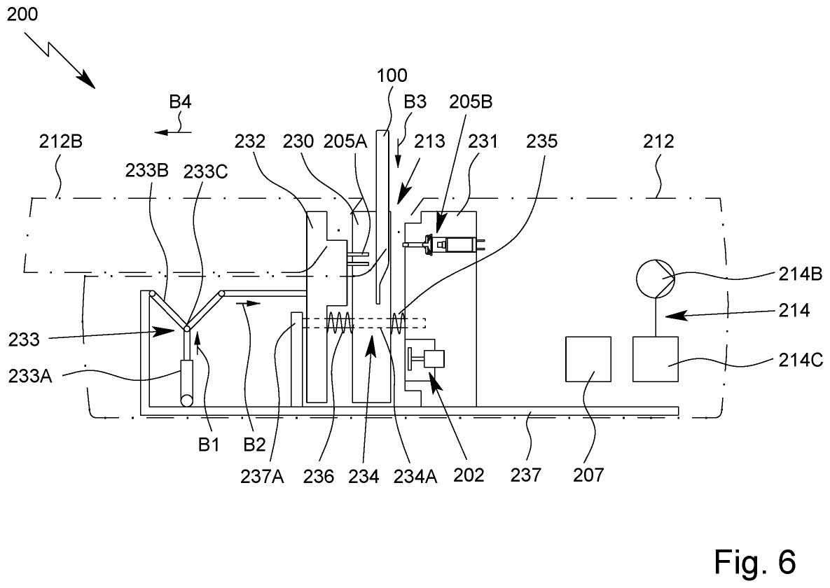

Preferably, in the operating position of the cartridge 100, the intermediate connection 104D is arranged so as to be higher than the outlet 104C and/or lower than the inlet 104B and/or lower than the connection 104A, as can be seen in FIG. 6 (if FIG. 6 is rotated counter-clockwise by 90°).

In the operating position, if necessary a supernatant of the sample P, such as blood serum or blood plasma from a blood sample, can be discharged or carried away via the intermediate connection 104D.

Preferably, the width J2 (shown in FIG. 2) and/or the depth J3 (shown in FIG. 1) of the receiving cavity 104 tapers towards the outlet 104C. This is conducive to effectively discharging the sample P in the operating position.

Preferably, as already explained, one initially closed valve 115A that is closed in the delivery state of the cartridge 100 is respectively assigned to each of the inlet 104B, the outlet 104C and, if it is provided, the optional intermediate connection 104D. These valves 115A are only opened by the analysis device 200 later, as required. This ensures that the sample P cannot flow into or flow away in other channels or cavities in an undesired or undefined manner following the filling process or during the filling process.

After the receiving cavity 104 has been filled with the sample P, the connection 104A is closed by the closure element 130 and/or the closure part 132 thereof being placed onto the connection 104A in order to sealingly or tightly close said connection.

The analysis device 200 and/or pressurized gas supply 214 preferably comprises a connection element 214A, in particular in order to pneumatically connect the analysis device 200 and/or pressurized gas supply 214 to the cartridge 100 and/or to the connection 129 or connections 129.

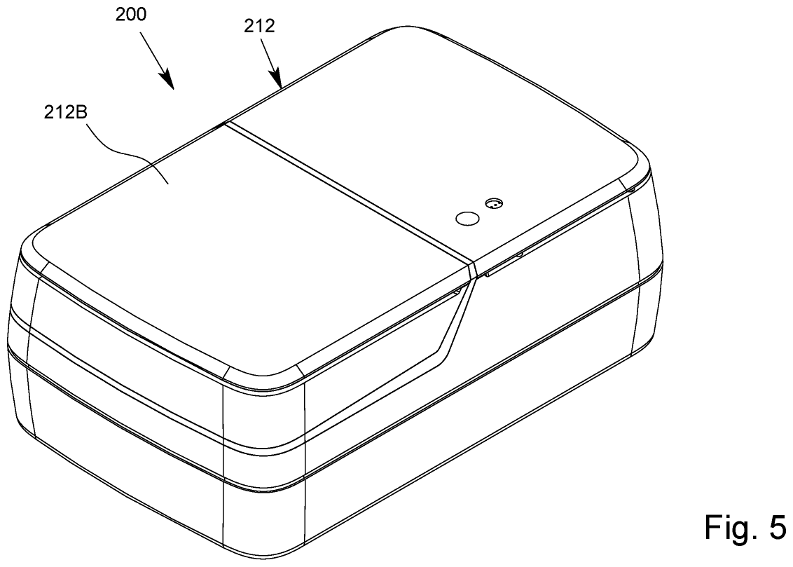

FIG. 5 is a perspective view of the proposed analysis device 200 in the closed state. The analysis device 200 or housing 212 preferably comprises a housing part 212B that can be opened.

FIG. 6 is a schematic section through the analysis device in the open state, i.e., when the housing 212 or housing part 212B is open. The opening 213 in the analysis device 200 or housing 212 is therefore open. In this view, the cartridge 100 is inserted into the analysis device 200 in part or received therein in part.

The analysis device 200 and/or pressurized gas supply 214 preferably comprises a compressor 214B, in order to compress, condense and/or pressurize the working medium, in particular gas or air, and optionally comprises an associated pressurized gas storage means 214C, as shown schematically in FIG. 6.

The analysis device 200 is designed to receive, position and/or hold the cartridge 100, in particular such that the cartridge 100 can be held in a clamped manner and/or can be mechanically, electrically, thermally, fluidically and/or pneumatically connected.

The analysis device 200 preferably comprises a receiving unit 230, which is used in particular to receive, position and/or hold the cartridge 100, a connection unit 231, which is used in particular to mechanically, electrically, thermally and/or fluidically connect the cartridge 100, an actuator unit 232 for actuating or forcing one or more valves 115A open, and/or a drive apparatus 233, in particular for moving or actuating the receiving unit 230 and/or actuator unit 232.

The analysis device 200 preferably comprises a pneumatically operated apparatus for holding, mounting, positioning and/or clamping the cartridge 100. In this case, said apparatus is formed in particular by the receiving unit 230, the connection unit 231, the drive apparatus 233, and optionally the actuator unit 232.

The analysis device 200 and/or drive apparatus 233 preferably comprises an in particular pneumatically operated drive 233A, such as a pneumatic cylinder, and/or a gear mechanism 233B. Preferably, the drive apparatus 233 or gear mechanism 233B is operated, actuated and/or driven pneumatically.

In the example shown, the gear mechanism 233B is preferably designed as a reduction gear mechanism and/or a gear mechanism having a variable reduction ratio, particularly preferably designed as a toggle lever mechanism. In particular, the drive 233A acts on the toggle link or joint 233C, as shown in FIG. 6, in order to convert the drive movement in the direction B1 into a driven or actuator movement in the direction B2. However, other structural solutions are also possible.

The direction B1 of the drive movement preferably extends transversely or at least substantially perpendicularly to the direction B2 of the actuator movement and/or an opening direction B4, and/or at least substantially parallel, but preferably in the opposite direction, to a receiving direction B3.

The analysis device 200 preferably comprises a guide apparatus 234 for movably and/or slidably guiding the receiving unit 230 and/or actuator unit 232 as shown schematically in FIG. 6. In particular, the guide apparatus 234 holds or guides the connection unit 230 and/or actuator unit 232 such that said connection unit and/or actuator unit can move and/or slide relative to the connection unit 231 and/or in the direction B2 or in the opposite direction.

In FIG. 6, the actuator movement in the direction B2 is the movement towards the connection unit 231, i.e., a closing movement or advancing movement.

In the view according to FIG. 6, the receiving unit 230 has been moved away from the connection unit 231. This constitutes the receiving position in which the cartridge 100 can be received and then ejected or removed. In FIG. 6, the cartridge 100 is partly received or slid in and is in a transfer position.

Once the receiving unit 230 has received the entire cartridge 100 and has been moved together therewith towards the connection unit 231, i.e., when advancement or closing has occurred, the cartridge 100 is positioned against or abuts the connection unit 231. In the following, this is also referred to as the test position of the receiving unit 230 and cartridge 100.

The receiving unit 230 is preferably biased or pretensioned into the receiving position, preferably by means of at least one spring 235.

The actuator unit 232 preferably comprises one or more actuators 205A, in particular in the form of fixed actuation elements or actuation pins, as shown schematically in FIG. 6. In the following, the moved-away or unactuated position of the actuator unit 232 as shown is also referred to as the initial position.

The actuator unit 232 is preferably biased or pretensioned into the initial position, preferably by means of at least one spring 236.

The actuator unit 232 can be moved out of the initial position by means of the drive apparatus 233 (relative to or) towards the connection unit 231 and/or receiving unit 230 and/or in the direction B2.

The guide apparatus 234 preferably comprises at least one guide element 234A, which is a guide rod in this case, for holding or guiding the actuator unit 232 and/or receiving unit 230 such that said actuator unit and/or receiving unit can in particular move linearly, in particular in the direction B2 of the actuator movement or in the direction opposite thereto.

The guide element 234A is preferably supported or mounted, at one end, on the connection unit 231 and/or is held or mounted, at the other end, on an abutment or rack 237 or stop 237A of the analysis device 200.

The analysis device 200 or the receiving unit 230 preferably comprises a lifting apparatus in order for it to be possible for the cartridge 100 to enter the receiving unit 230 in a preferably vertical direction or in a receiving direction B3 or receiving movement and/or in order for it to be possible for said cartridge to be ejected from or moved out of said unit in the opposite direction and/or towards the top.

The receiving direction B3 preferably extends transversely and/or perpendicularly to the direction B2 of actuator movement or advancement movement.

FIG. 6 shows the cartridge 100 in its transfer position. In said transfer position, the cartridge 100 that has not yet been used is manually inserted into or transferred to the analysis device 200.

From the transfer position, the cartridge 100 is lowered and/or moved, by means of the lifting apparatus, into a position in which it is in a lower position and/or is received in its entirety in the receiving unit 230, as indicated by movement in the receiving direction B3.

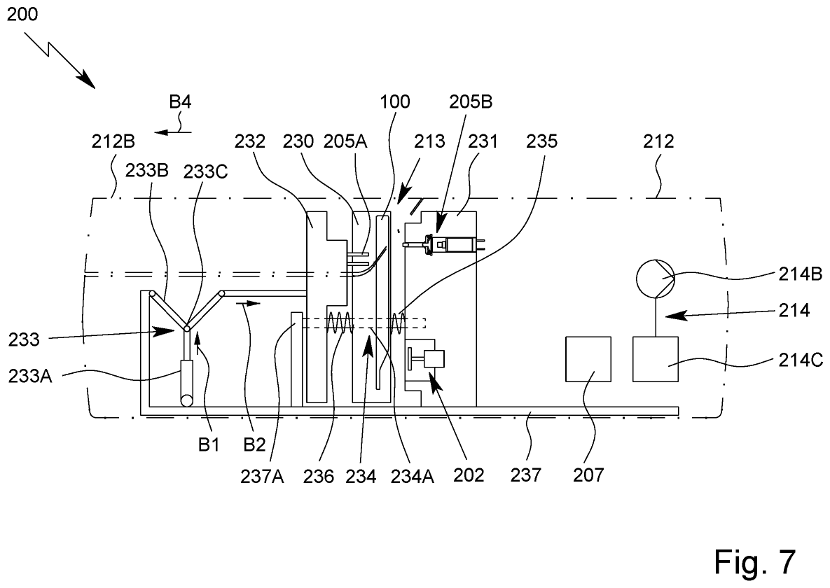

FIG. 7 is a schematic section through the cartridge 100 that corresponds to the section in FIG. 6, in which the cartridge is in its position in which it has been received in its entirety, the actuator unit 232 (still) being in the initial position and the receiving unit 230 (still) being in the receiving position, and the analysis device 200 or housing 212 or housing part 212B (already) being closed.

In order to close the analysis device 200 or the (outer or housing-side) opening 213 in the analysis device 200, in the example shown, the housing part 212B is moved or closed in a direction opposite to an opening direction B4.

The opening direction B4 preferably extends horizontally and/or in parallel with the direction B2 of the actuator movement or advancement movement.

The opening direction B4 preferably extends transversely and/or perpendicularly to the receiving direction B3.

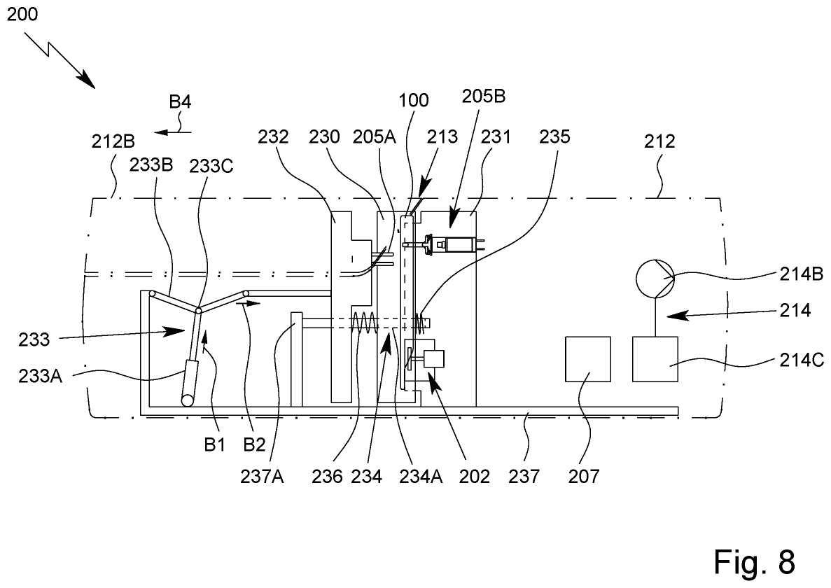

Once the cartridge 100 has been received or brought in in this manner and the housing 212 of or the opening 213 in the analysis device 200 has preferably been closed, the cartridge 100 or the receiving unit 230 containing the cartridge 100 is moved, in a first step or period of movement, preferably towards the connection unit 231, in particular until the connection unit 231 and the cartridge 100 are connected in the desired manner and/or are in abutment in the desired manner and/or until the cartridge 100 is positioned on or against the connection unit 231 in the desired manner and/or until the cartridge 100 is clamped between the connection unit 231 and the receiving unit 230 in the desired manner, i.e., until the receiving unit 230 and thus also the cartridge 100 have reached the test position. This state is shown in the schematic section according to FIG. 8 that corresponds to the section in FIGS. 6 and 7.

Thus, the receiving unit 230, the connection unit 231 and/or the actuator unit 232 preferably can be moved relative to each other and/or along the direction B2 of the actuator movement (back and forth). In particular, the receiving unit 230 and/or the actuator unit 232 can be moved in the direction of and/or relative to the connection unit 231, as explained in greater detail in the following.

In the example shown, the connection unit 231 is preferably fixed and/or immovable and/or stationary, in particular allowing a simple construction. However, it is also possible to design the connection unit 231 so as to be movable, for example similar or alternatively to the receiving unit 230.

In the state shown in FIG. 8, the actuator unit 232 has preferably not yet been moved relative to the receiving unit 230, but has preferably already been moved relative to the connection unit 231. This position of the actuator unit 232 is also referred to as the intermediate position.

Preferably, when the receiving unit 230 or cartridge 100 is being transferred or moved into the test position and/or preferably immediately thereafter, at least one valve 115A of the cartridge 100 is actuated and/or opened in an automatic and/or forced manner Particularly preferably, a plurality or all of the valves 115A that, in principle, have to be actuated and/or opened for the test and/or that require a particularly high actuation force are actuated or opened in a forced manner These valves are actuated in particular by means of the actuator unit 232.

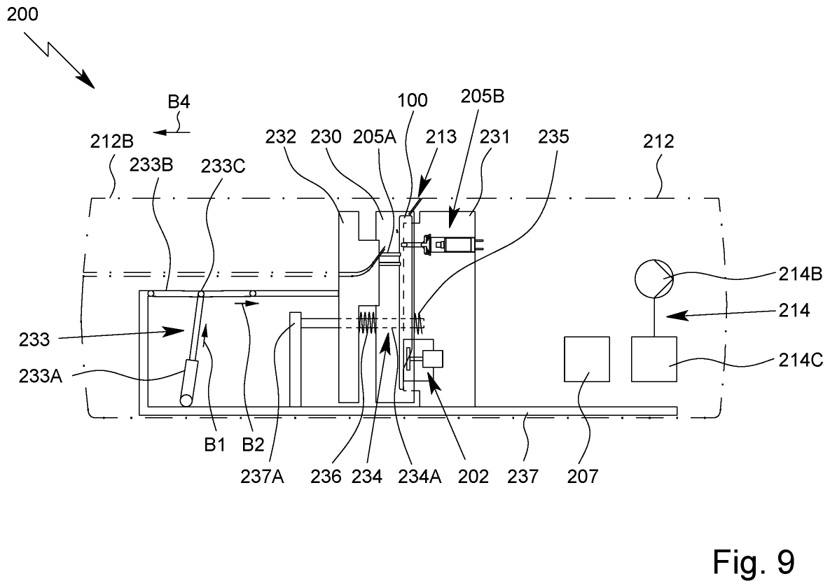

In a second step or period of movement or immediately after the test position has been reached, the actuator unit 232 is preferably moved relative to or into the receiving unit 230 such that the actuator unit 232 ultimately assumes an actuation position, as indicated in FIG. 9, in which position the actuator unit 232 actuates, particularly preferably forces open, the corresponding valves 115A of the cartridge 100 by means of the actuators 205A of said actuator unit 232.

The purpose of the first movement or the first step is in particular to receive the cartridge 100 in a clamped manner, to position the cartridge 100, to firmly mount the cartridge 100 and/or to connect the cartridge 100.

The purpose of the second movement or the second step is in particular to actuate or open preferably a plurality of valves 115A of the cartridge 100. However, the actuator unit 232 can, alternatively or additionally, also be used for other purposes or forms of actuation and/or can be used to connect the cartridge 100 in further or other ways.

In the state shown in FIG. 9, the sample P is then tested. In this state, the cartridge 100 is connected in the necessary manner to the analysis device 200 or vice versa. However, the test can, in principle, also start in an earlier state, in particular as shown in FIG. 8, for example when it is not necessary to actuate valves 115A, when it is only optionally necessary to actuate said valves or when it is only necessary to actuate said valves in the (further) test sequence.

It is noted that, in the example shown, the actuator movement is preferably at least substantially parallel to the advancement movement by means of which the receiving unit 230 and/or actuator unit 232 is/are moved towards the connection unit 231, in particular both movements being in the direction B2. However, in principle, these movement directions can also extend obliquely to one another.

The drive apparatus 233 preferably acts on the receiving unit 230 either directly or indirectly.

In the example shown, the drive apparatus 233 preferably acts on the receiving unit 230 only indirectly, since the drive apparatus 233 engages in particular on the actuator unit 232 and acts on the receiving unit 230 by means of said actuator unit or a spring coupling, in particular in order to bring about the desired advancement movement in the direction B2 or movement relative to the connection unit 231.

The actuator unit 232 is thus used for moving the receiving unit 230 relative to the connection unit 231, in particular in order to position, place or clamp the cartridge 100 on the connection unit 231, and/or for actuating or opening one or more valves 115A of the cartridge 100.

Preferably, the actuator unit 232 and the receiving unit 230 can be moved together in the first period of movement or step and can be moved relative to one another in the movement direction B2 in the second period of movement or step, in particular in order to move the cartridge 100 towards the connection unit 231 and in particular also in order to open one or more valves 115A.

The actuator unit 232 can preferably be moved counter to or against a spring force, in this case counter to or against the force of the spring(s) 235, 236, towards the receiving unit 230 and/or connection unit 231.

Preferably, just one single or common drive apparatus 233 is provided in order to move or slide the receiving unit 230 and the actuator unit 232. This provides for a particularly simple, compact and/or robust construction.

Particularly preferably, the actuator unit 232 and the receiving unit 230 perform a coupled movement, i.e., are motion-coupled, in this case by the springs 235 and 236.

However, some other type of coupling, for example that uses gears, levers or a slotted link or the like, can also be used or carried out.

The drive apparatus 233 preferably operates in one direction, in this case in the closing direction B2, counter to or against a spring force. The opposite movement or return movement into the initial position can be performed in particular only by the spring force. This allows, for example, for a single-acting cylinder to be used as the drive 233A.

The spring force by means of which the receiving unit 230 can be moved towards the connection unit 231 is preferably smaller than the spring force by means of which the actuator unit 232 can be moved towards the receiving unit 230. Therefore, the desired coupled movement or the desired sequence of movements can be achieved in a simple manner, particularly preferably such that the cartridge 100 is initially positioned on, connected to and/or clamped on the connection unit 231 and only after this are one or more valves 115A of the cartridge 100 actuated or opened in a forced manner

However, the coupled movement or the sequence of the two steps can also be achieved or solved by means of a different structure, optionally by means of just one single or common drive apparatus 233, as is the case in the preferred embodiment, or, alternatively, by means of separate drive apparatuses for the different movements or steps.

The receiving unit 230 can preferably be moved relative to the connection unit 231 by means of a motor, in particular pneumatically.

The actuator unit 232 can preferably be moved relative to the connection unit 231 and/or receiving unit 230 by means of a motor, in particular pneumatically.

Valves 115A on the cartridge 100 are actuated in particular in a mechanical manner by the actuator unit 232 acting on the cartridge 100 or the valves 115A thereof, in the actuation position, by means of actuators 205A that are preferably fixed on the actuator unit 232. In this regard, the actuators 205A can optionally engage through apertures or through holes in the main body 101 if the valves 115A are arranged on the flat side of the cartridge 100 that is remote from the actuator unit 232, as is the case in the example shown.

The front 100A or the cover 102 of the cartridge 100 preferably points towards the receiving unit 230.

In particular, the receiving unit 230 has a contact surface for the cartridge 100, in particular the front 100A or cover 102 thereof, that is at least substantially planar, flat and/or continuous, in order to support the cartridge 100 in the test position in as even a manner as possible and/or over the largest possible surface area and/or in order to hold and/or clamp said cartridge against the connection unit 231.

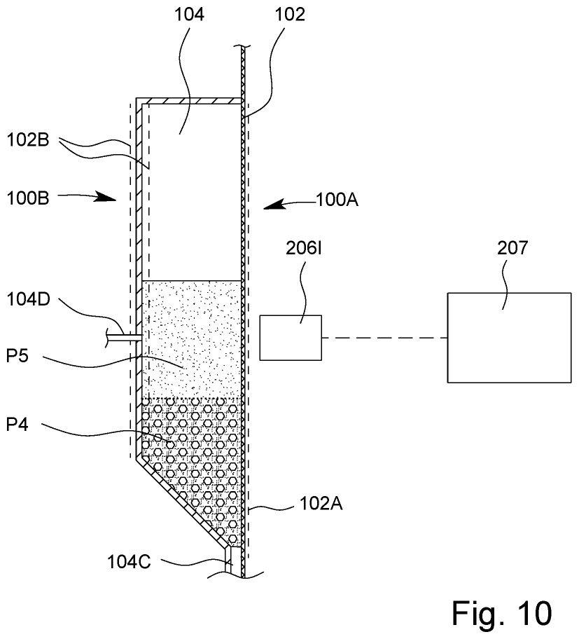

In the example shown, the actuator unit 232 preferably comprises a plurality of fixed actuators 205A, in particular two groups of actuators 205A (right-hand side and center of FIG. 10) which actuate or open associated valves 115A of the cartridge 100 in the actuation position preferably in a forced manner These actuators 205A and/or valves 115A are in particular assigned to the storage cavities 108 in order to open said cavities.

Also large opening forces can be achieved by means of the drive apparatus 233, and therefore also valves 115A that close in a particularly tight manner and ensure a high level of tightness and thus also high storage stability can be used and initially opened.

The actuator unit 232 or the receiving unit 230 preferably comprises actuators 205A that can be actuated independently of the movement of the receiving unit 230 relative to the connection unit 231, independently of the movement of the actuator unit 232 towards the receiving unit 230 and/or independently of one another, which actuators are preferably in the form of three adjacent pairs of pins, and are used in particular for opening the valves 115A assigned to the receiving cavity 104 or other valves, as required. Said actuators 205A comprise separate drives (not shown) for individual actuation. The inlet 104B, outlet 104C and intermediate connection 104D can thus be opened as required and on an individual basis.

The connection unit 231 forms in particular an abutment or a contact surface for the cartridge 100 in the test position. In particular, the connection unit 231 comprises for this purpose corresponding contact surfaces or support regions that support the cartridge 100 in the test position, preferably on the back 100B thereof.

The cartridge 100 is preferably positioned in a defined manner in the test position. This can be achieved in particular by means of corresponding engagement with the receiving unit 230 and/or the connection unit 231.

The pump drive 202 or the pump motor 202A thereof and the temperature-control apparatuses 204 are preferably operated electrically and in particular supplied with electrical power by the power supply 211 and/or controlled by the control apparatus 207.

A plurality of apparatuses of the analysis device 200, such as the drive apparatus 233, the actuators 205B and/or the means for supplying pressurized working medium via the connection elements 214A, are preferably controlled and/or operated by the control apparatus 207 by activating corresponding valves and correspondingly supplying pressurized gas or pressurized air from the pressurized gas supply 214.

The analysis system 1 preferably comprises a sample sensor 206I for monitoring the sample P, in particular in the receiving cavity 104 of the cartridge 100. Preferably, the analysis device 200, in particular the receiving unit 230 and/or the connection unit 231, comprises the sample sensor 206I. However, it is also possible that the cartridge 100 comprises the sample sensor 206I or at least parts thereof.

The sample sensor 206I is schematically displayed in FIGS. 1 and 10.

It is possible that the sample sensor 206I comprises different parts or portions which are arranged on or attached to different parts of the analysis device 200 and/or the cartridge 100. For example, the sample sensor 206I can comprise a part which is arranged in or on the receiving unit 230 and a further part which is arranged in or on the connection unit 231 and/or the cartridge 100.

In particular, the further cover 102B of the receiving cavity 104 can be or form a part of the sample sensor 206I.

It is preferred that testing of the sample P or pumping the sample P out of the receiving cavity 104 is not performed immediately after introduction of the sample P into the receiving cavity, but that the sample P is pretreated or prepared for the subsequent test within the receiving cavity 104. In particular, pretreating or preparing of the sample P involves separation and/or sedimentation of the sample P.

In particular, sedimentation or separation of the sample P is the process of separating different components, in particular a solid and a liquid phase of a fluid or suspension, such as blood. Blood comprises solid components or a solid phase, in particular blood cells, and liquid components or a liquid phase, in particular blood serum or blood plasma, which are normally mixed or form a suspension and can be separated from one another.

“Components” of the sample P are in particular the solid and liquid phases or components of the sample which are separated by sedimentation.

FIG. 10 shows the receiving cavity 104 with the sample P after sedimentation.

The sample P preferably comprises at least two phases or components, namely a first component P4 and a second component P5. Preferably, the first component P4 is solid and/or the second component P5 is liquid. The components P4, P5 are displayed in FIG. 10.

Preferably, the sample P is blood, wherein blood cells form the first component P4 and blood plasma or blood serum forms the second component P5.

Typically, the liquid phase will be above the solid phase after sedimentation. The liquid phase is therefore also called supernatant.

The sample sensor 206I or the analysis device 200 by means of the sample sensor 206I is preferably designed to monitor, measure or detect the process and/or the result of sedimentation.

The monitoring, measuring or detecting by the sample sensor 206I or analysis device 200 can be performed continuously or discretely. It is in particular possible to perform measurements with the sample sensor 206I in discrete intervals, for example every few seconds or minutes.

The sample sensor 206I or the analysis device 200 by means of the sample sensor 206I is preferably designed to measure or detect the filling level of the sample P in the receiving cavity 104, in particular the filling level of each of the separated components P4, P5 and/or the phase boundary or interface between separated components P4, P5.

Preferably, the sample sensor 206I or the analysis device 200 by means of the sample sensor 206I is designed to measure or detect a degree of separation of the sample P or components P4, P5 in the receiving cavity 104.

In particular, the sample sensor 206I or the analysis device 200 by means of the sample sensor 206I is designed to detect a finished or sufficient sedimentation or separation of the sample P. Preferably, it is possible to measure or detect with the sample sensor 206I, whether a desired, predefined or predetermined value or threshold, which in particular is related to or corresponds to a degree of separation or sedimentation, has been reached.

In particular, the sample sensor 206I or the analysis device 200 by means of the sample sensor 206I is designed to measure or detect a supernatant of the sample P or whether a supernatant has (already) formed.

The sample sensor 206I is preferably designed to measure or detect a density, a transparency, a cloudiness, a color and/or a viscosity of the sample P or of at least one of the components P4, P5, in particular the separated phases or components. Preferably, the at least one or more of the named quantities differ between the (separated) components P4, P5 of the sample P or between the sample P as introduced in the receiving cavity 104 and after sedimentation. In particular, at least one of the named quantities change during sedimentation of the sample P. Thus, measuring or detecting the sedimentation or a progress thereof is made possible by measuring or detecting the named quantities.

In particular, the first component P4, in particular blood cells, is denser, less transparent, cloudier, darker and/or more viscous than the second component, in particular blood serum or blood plasma. The first component P4 is preferably solid, impervious to light and/or of a dark reddish color. The second component P5 is preferably liquid, clear, highly previous to light, and/or of a bright color or at least substantially colorless.

As an alternative or in addition, the sample sensor 206I is preferably designed to measure or detect a temperature of the sample P or of at least one of the components P4, P5.

Preferably, the sample sensor 206I is designed to detect or measure electromagnetic radiation, in particular visible light and/or infrared radiation. The sample sensor 206I is or comprises in particular an optical or infrared sensor or is designed to detect electromagnetic radiation with a wave length of at least approximately 380 nm and/or at most approximately 10 μm.

The sample sensor 206I preferably comprises a generator or transmitter for generating and/or transmitting electromagnetic, in particular optical or infrared, waves or radiation and/or a receiver for receiving, measuring and/or detecting in particular reflected electromagnetic, in particular optical or infrared, waves or radiation. Further, the sample sensor 206I can comprise a reflector for reflecting the generated and/or transmitted electromagnetic waves or radiation.

According to a preferred embodiment, the further cover/layer 102B on the back of the receiving of the receiving cavity 104 forms a part of the sample sensor 206I and/or serves as or forms the reflector for reflecting radiation or light generated by the sample sensor 206I.

As an alternative or in addition, the connection unit 231 or the receiving unit 230 could form or comprise the reflector.

The sample sensor 206I can be—as an alternative or in addition—designed to measure, detect or monitor the sample P by a transmission measurement.

The sample sensor 206I can be arranged on two different or opposite sides of the cartridge 100 or the receiving cavity 104, in particular the front 100A and the back 100B. In particular, the sample sensor 206I comprises a part arranged on the front 100A and a part arranged on the back 100B.

Preferably, the receiving cavity 104 is on at least one side, preferably on two sides, provided with or covered by a material, in particular a foil, which is transparent or previous to the radiation transmitted, received and/or measured by the sample sensor 206I. For example, the receiving cavity 104, main body 101 and/or further cover 102B can comprise a through hole, in particular on the back 100B, which is covered by said transparent material or foil. The transparent material or foil is preferably made of at least substantially the same material as the cover 102.

The receiving cavity 104 preferably comprises a window which can be passed by radiation or light to be transmitted, received and/or measured by the sample sensor 206I. Particularly, said window is formed by said through hole and/or the material covering said through hole.

A transmission measurement of the sample P in the receiving cavity 104 can for example be performed by a transmitter of the sample sensor 206I which is positioned on one side of the receiving cavity 104 and a receiver of the sample sensor 206I which is positioned on the other side of the cavity so that radiation transmitted by the transmitter through the sample P can be received or measured by the receiver and, preferably, subsequently analyzed.

Measuring or detecting by the sample sensor 206I can be performed by analyzing the properties of received or detected radiation or light, in particular an intensity, a brightness, a wave length, a direction or the like. As an alternative or in addition, radiation or light generated by the sample sensor 206I can be compared to the radiation or light received by the sample sensor 206I in order to gain information about the sample P. In particular, absorption of the radiation or light or certain spectral ranges thereof by the sample P can be measured or detected.

As an alternative or in addition, the sample sensor 206I can work capacitively. In this case, the further cover/layer 102B can in particular serve as a part or plate of a capacitor, in particular in order to measure or detect the capacity of the sample P or of at least one of the components P4, P5 or a change in the capacity.

With a capacitive sensor, it is in particular possible to differentiate the components P4, P5 based on their dielectric constants or properties which are preferably different.

The sample sensor 206I is preferably arranged in a position which allows to measure or detect one of the components P4, P5, both components P4, P5 and/or the phase boundary or interface between the components P4, P5, as desired. In particular, the sample sensor 206I can comprise a plurality of sensors or detectors, for example in the form of a sensor array, in particular for separately measuring the components P4, P5 and/or for measuring or monitoring the sample P at different filling levels in the receiving cavity 104.

In particular, in the case of a plurality of sensors or detectors which are arranged at different positions or filling levels along the receiving cavity 104, it is possible that the analysis device 200 or sample sensor 206I is configured to evaluate (only) the difference in the signals measured by the different sensors or detectors, preferably so that it can be detected when the components P4, P5 have separated. In this way, for example, a difference in transparency, color and/or dielectric constant between the components P4, P5 could be easily measured and/or the components P4, P5 could be easily differentiated.

The analysis device 200 is preferably designed to, in particular, automatically place the sample sensor 206I or its sensor and/or the cartridge 100 in an operating position, preferably upon or after insertion of the cartridge 100, so that the sample P in the receiving cavity 104 can be monitored by the sample sensor 206I in the operating position.

The sample sensor 206I or at least a part thereof is preferably arranged in or on the receiving unit 230 in a fixed position relative to the receiving unit.

The operating position is, in particular, a position of the sample sensor 206I and the cartridge 100 or receiving cavity 104 relative to one another which allows monitoring of the sample P in the receiving cavity 104 with the sample sensor 206I. The operating position is in particular indicated in FIGS. 1 and 10.

Preferably, the sample sensor 206I in the operating position is at least essentially aligned to or arranged in the same height or vertical position as the intermediate connection 104D. However, it is also possible to locate the sample sensor 206I in another position, for example (slightly) above or below the intermediate connection 104D and/or in the area of an (expected) phase boundary or interface between the components P4, P5.

In particular, the location of the sample sensor 206I in the operating position is matched or coordinated with a (typical or average) volume of the sample P and/or the components P4, P5 or a filling level thereof in the receiving cavity 104.

The sample sensor 206I is preferably arranged or located (in the operating position) at the front 100A and/or flat side of the cartridge 100 or on the side of the cartridge 100 covered by or comprising the cover 102.

The cover 102 is preferably transparent or previous to radiation generated and/or received, measured or detected by the sample sensor 206I, in particular (visible) light and/or infrared radiation.

The cartridge 100 and/or sample sensor 206I can preferably be arranged or positioned in the operating position by a respective movement of the connection unit 231 and/or receiving unit 230, in particular as described above.

The sample sensor 206I is preferably coupled or connected to the analysis device 200 or in particular to the control apparatus 207, in particular by a data connection. The data connection is indicated by a dashed line in FIGS. 1 and 10. The signals created or measured by the sample sensor 206I can preferably be transmitted to the control apparatus 207, in particular via the data connection.

The control apparatus 207 which has been described before is preferably designed for in particular automatically starting and/or controlling testing or further processing of the sample P on the basis of signals received from the sample sensor 206I.

In particular, the control apparatus 207 is designed to analyze, evaluate and/or interpret the signal(s) received from the sample sensor 206I and/or to start and/or control testing of the sample P or component(s) P4, P5 in response to the signal(s) or measured/determined state of the sample P.

The control apparatus 207 is preferably designed to compare the signals received from the sample sensor 206I to a desired, predetermined or predefined value or threshold, in particular so that it can be decided whether sufficient or complete sedimentation or preparation of the sample P has been reached.

In the proposed method, the sample P is preferably introduced in the receiving cavity 104 of the cartridge 100.

The cartridge 100 is preferably introduced or inserted into the analysis device 200 or received by the analysis device 200, in particular after introduction of the sample P in the cartridge 100.

When the cartridge 100 is (correctly) positioned in the analysis device 200, preferably sedimentation or separation of the sample P takes place or is performed. This can be done for example by simply waiting until different phases of the sample P separate due to gravity. However, it is also possible to accelerate the process of sedimentation, for example by centrifugation.

Sedimentation or preparation of the sample P is preferably performed immediately after introducing the sample P into the cartridge 100 and/or receiving cavity 104 and/or as a first step of a method of testing the sample P. Preferably, amplification, temperating and/or detecting or measuring the sample takes place after sedimentation and/or in a cavity, channel or apparatus which is different from the receiving cavity and/or separated or separable from the receiving cavity 104 by one or more valves 115.

The sample P, in particular the process and/or progress of sedimentation thereof, is preferably monitored by means of the sample sensor 206I.

Further processing of the sample P, such as discharging the sample P or the components P4, P5 from the receiving cavity 104 for testing the sample P and further subsequent steps, are preferably based on signals of the sample sensor 206I which are in particular transmitted to the control apparatus 207, as explained above.

Testing of the sample P or discharging the sample P from the receiving cavity 104 is preferably only started when complete or sufficient sedimentation/preparation of the sample P has been measured or detected by the sample sensor 206I, in particular when a value measured or detected by means of the sample sensor 206I reaches or exceeds a predetermined or predefined value or threshold.

In particular, the sample P is kept in the receiving cavity until the sample sensor 206I detects a supernatant or sufficient or complete sedimentation of the sample P. In other words, the outlet 104C and/or the intermediate connection 104D is kept closed and/or (only) opened or opened as soon as the sample sensor 206I detects a supernatant or a sufficient or complete sedimentation of the sample P.

After sedimentation, one, several or all separated parts, phases or components P4, P5 of the sample P can be tested or used for further analysis. It is also possible, that the separated components P4, P5 are separately tested or analyzed in the cartridge 100, in particular one after another.

The same or different assays or procedures can be performed on different components P4, P5 of the sample P. Alternatively, only one of the components P4, P5 is tested or analyzed and/or the other component P4, P5 is not tested or analyzed, in particular wherein the other component P4, P5 is conveyed to the collection cavity 111.

The sample P is preferably removed or discharged from or pumped out of the receiving cavity 104 by the pump apparatus 112, in particular after a supernatant or sufficient or complete sedimentation of the sample P is detected and/or before further processing steps are performed.

In particular, one or more of the following steps are performed outside and/or downstream of the receiving cavity 104 and/or after a supernatant or sufficient or complete sedimentation of the sample P is detected and/or after the sample P has been removed from the receiving cavity 104: Amplification, testing, detecting and/or identifying of the sample P or components thereof, in particular performing PCR, carrying out a nucleic-acid assay, carrying out a protein assay and/or carrying out electrochemical detection.

As explained before, the receiving cavity 104 preferably comprises more than one outlet/connection 104C/104D for discharging or removing the sample P or the components P4, P5 from the receiving cavity 104. In particular, the receiving cavity 104 comprises the outlet 104C and the intermediate connection 104D, as described above.

Preferably, the separated phases or components P4, P5 are discharged from the receiving cavity 104 via different connections. In particular, the solid or lower component P4 of the sample P is discharged via the outlet 104C and/or the upper or liquid component P5 is discharged via the intermediate connection 104D.

In a particularly preferred example, blood cells are discharged or removed from the receiving cavity 104 via the outlet 104C and/or blood serum or blood plasma is discharged or removed from the receiving cavity 104 via the intermediate connection 104D.

It is also possible, however, to first discharge the lower, in particular solid, component P4 via the outlet 104C and to discharge the liquid or upper component P5 via the outlet 104C thereafter, in particular after testing of the solid or first component P4 has been completed.

The components P4, P5 can be lead or conveyed through the cartridge 100 or fluidic system 103 along the same or along different paths, as desired or needed.

According to a further aspect, it is possible to perform further steps of pretreatment and/or preparation of the sample P. This can be done before or after discharging the sample P from the receiving cavity 104, as desired. In particular, one or more of the steps or methods listed in the following take place or are performed in one or more of the cavities 105-110.

Pretreatment and/or preparation of the sample is preferably performed for one or both components P4, P5, preferably independently.

In particular, pretreatment comprises cell disruption, for example by a chemical, mechanical and/or biological, in particular enzymatic, method.

Further, in particular in addition to cell disruption, pretreatment and/or testing the sample P can comprise plasma fractionation, plasma sequencing, cell fractionation, protein purification and/or DNA isolation, as desired or needed.

Individual aspects and features of the present invention and individual method steps and/or method variants may be implemented independently from one another, but also in any desired combination and/or order.

1-41. (canceled)

42. An analysis device for testing a sample by means of a cartridge, comprising:
a holder for a cartridge,
a sensor apparatus with a sample sensor for monitoring the sample in a receiving cavity of the cartridge, the sample sensor being adapted to detect a supernatant or sedimentation of the sample,
means for at least one of electrically, thermally, fluidically or pneumatically connecting the cartridge to the sensor apparatus, and
a pump drive for pumping the sample or supernatant to the sensor apparatus or catcher molecules of the cartridge after the supernatant or sedimentation of the sample has been sensed by the sample sensor.

43. The analysis device according to claim 42, wherein the supernatant is blood plasma or blood serum.

44. The analysis device according to claim 42, wherein the sample sensor is adapted to detect at least one of visible light or infrared radiation.

45. The analysis device according to claim 42, wherein the sample sensor is adapted to monitor the sample by a reflection measurement.

46. The analysis device according to claim 42, wherein the holder is adapted to automatically place at least one of the sample sensor or the cartridge in an operating position upon or after insertion of the cartridge so that the sample in the receiving cavity is able to be monitored by the sample sensor in the operating position.

47. The analysis device according to claim 42, further comprising a control apparatus for automatically at least one of starting or controlling testing of the sample based on signals received from the sample sensor.

48. The analysis device according to claim 43, wherein the holder comprises a receiving unit for at least one receiving, positioning or holding the cartridge.

49. A cartridge for testing a sample, comprising:
a main body having a plurality of channels and cavities,
a cover for the channels and cavities,
a receiving cavity for receiving the sample,
a sample sensor or a part thereof for detecting sedimentation or a supernatant of the sample in the receiving cavity, and
a nucleic-acid assay or protein assay.

50. The cartridge according to claim 49, wherein the receiving cavity is arranged upstream of the assay.

51. An analysis system for testing a sample, comprising:
a cartridge for receiving and testing the sample,
an analysis device adapted to hold the cartridge at least one of electrically, thermally, or pneumatically connect thereto,
wherein the cartridge comprising a sensor array with different catcher molecules for detecting analytes of the sample, and a receiving cavity for the sample,
the analysis device comprises a sample sensor for monitoring the sample in the receiving cavity, and
further comprising a pump for pumping a supernatant of the sample for further processing in the cartridge and later detection of analytes after the sample sensor has detected the supernatant or sedimentation of the sample in the receiving cavity.

52. The analysis system according to claim 51, wherein the sample sensor is adapted to detect at least one of visible light or infrared radiation.

53. The analysis system according to claim 51, wherein the sample sensor is adapted to monitor the sample by a reflection measurement.

54. Analysis system according to claim 51, wherein the analysis device is adapted to automatically place at least one of the sample sensor or the cartridge in an operating position upon or after insertion of the cartridge so that the sample in the receiving cavity can be monitored by the sample sensor in the operating position.

55. A method for testing a sample, with an analysis system comprising an analysis device, and a cartridge, comprising:
introducing a sample into a receiving cavity of the cartridge,
monitoring the sample in the receiving cavity with a sample sensor,
further processing of the sample based on signals from the sample sensor, and
keeping the sample in the receiving cavity until a supernatant or sedimentation of the sample is detected by the sample sensor.

56. The method according to claim 55, wherein sedimentation of the sample is performed immediately after introducing the sample into the cartridge.

57. The method according to claim 55, wherein the sample is pumped out of the receiving cavity by a pump apparatus after a supernatant or sedimentation of the sample is detected.

58. The method according to claim 55, wherein the sample or individual components of the sample are amplified and tested, detected or identified after at least one of a supernatant or sedimentation of the sample is detected or after the sample has been removed from the receiving cavity.

59. The method according to claim 55, wherein a nucleic-acid assay for detecting or identifying a target nucleic-acid sequence is carried out at least one of after a supernatant or sedimentation of the sample is detected or after the sample has been removed from the receiving cavity.

60. The method according to claim 55, wherein a protein assay for detecting or identifying a target protein is carried out at least one of after a supernatant or sedimentation of the sample is detected or after the sample has been removed from the receiving cavity.

61. The method according to claim 55, wherein electrochemical detection is carried out, at least one of after a supernatant or sedimentation of the sample is detected or after the sample has been removed from the receiving cavity.
